(19) United States
(12) Patent Application Publication
(10) Pub. No.: US 2025/0011874 A1
(43) Pub. Date: Jan. 9, 2025

(54) BREAST CANCER PROGNOSIS AND STRATIFICATION

(71) Applicant: Vestlandets Innovasjonsselskap AS, Bergen (NO)

(72) Inventors: Lars A. Akslen, Bergen (NO); Silje Kjølle, Bergen (NO); Kenneth Finne, Bergen (NO); Dimitrios Kleftogiannis, Bergen (NO)

(21) Appl. No.: 18/521,691

(22) Filed: Nov. 28, 2023

(30) Foreign Application Priority Data
Jul. 3, 2023 (GR) 20230100537

(51) Int. Cl. C12Q 1/6886; G01N 33/574

(52) U.S. Cl. CPC C12Q 1/6886; G01N 33/57415; C12Q 2600/112; C12Q 2600/118

(57) ABSTRACT

The present invention relates to methods of obtaining indications of the prognosis of breast cancer subjects, for classifying breast tumours and for predicting the therapeutic effectiveness of radiotherapy treatment on a subject with breast cancer. The methods are based on the production of a signature score which is derived from expression levels of a plurality of specific protein or RNA biomarkers.

The present invention relates to methods of treating breast cancer in a subject, obtaining indications of the prognosis of breast cancer subjects, for classifying breast tumours and for predicting the therapeutic effectiveness of radiotherapy treatment on a subject with breast cancer. The methods are based on the production of a signature score which is derived from normalised expression levels of a plurality of specific protein or RNA biomarkers.

Breast cancer is cancer that develops from breast tissue. Outcomes for breast cancer vary depending on the cancer type and subtype, by morphology and molecular profiles, the extent of disease, and the person's age. The five-year survival rates in England and the United States are between 80 and 90%. In developing countries, five-year survival rates are lower. Worldwide, breast cancer is the leading type of cancer in women, accounting for 25% of all cases. In 2018, it resulted in 2 million new cases and 627,000 deaths. It is more common in developed countries and is more than 100 times more common in women than in men.

There is therefore a significant need for new methods to diagnose breast cancer.

Reduced oxygen availability is a tumor microenvironment (TME) condition promoting cancer progress¹. Hypoxia-inducible factor 1-alpha (HIF-1α) accumulates and leads to a range of adaptive processes, such as metabolic changes, tumor plasticity, immune evasion, angiogenesis, and metastasis². Multiple target genes for HIF-1α have been reported, although cells may respond to hypoxia not exclusively through HIFs³. The complexity of hypoxia responses in human cancer tissues are not well studied at the proteomic level.

Intra-tumoral hypoxic regions often emerge as tumors outgrow their vascular supply, and hypoxia can trigger mechanisms like metabolic reprogramming and angiogenesis in the TME^{4,5,6} As an example, tumor vascular proliferation is linked to more aggressive subgroups of breast cancer⁷. Thus, hypoxia might represent a master regulator of several programs involved in tumor progression.

The Applicant has investigated hypoxia responses in the breast cancer TME by combining cell secretomes (in vitro) with the tumor stromal proteome (in vivo), with particular attention to differences between luminal-like and basal-like tumor subtypes. This integrated approach of secretome and stromal analysis has revealed a number of distinct proteomic patterns.

The Applicant has recognized that these proteomic patterns may be used in the prognosis and diagnosis of breast cancer.

There have been previous attempts to characterize breast cancers at both morphologic and molecular levels. Previously, Oncotype DX (van de Vijver M J et al. “A gene-expression signature as a predictor of survival in breast cancer”. N Engl J Med 2002; 347:1999-2009) and PAM50 (Parker J S et al., “Supervised risk predictor of breast cancer based on intrinsic subtypes”. J Clin Oncol 2009; 27:1160-7) have been used to classify breast tumors to inform prognosis and guide treatment. Oncotype DX is based on a panel of 16 cancer-related genes. PAM50 is a 50-gene signature that classifies breast cancer into five molecular intrinsic subtypes: Luminal A, Luminal B, HER2-enriched, Basal-like, and Normal-like. Each of the five molecular subtypes varies by their biological properties and prognoses. Luminal A generally has the best prognosis; HER2-enriched and Basal-like are considered more aggressive diseases.

However, the PAM50 and Oncotype DX expression signatures focus on the tumor cell compartment. They do not focus the tumor stroma.

The invention aims to overcome one or more of the above-mentioned problems or limitations by providing prognostic and diagnostic methods based on proteomic patterns of biomarkers which have been obtained from cell secretomes and tumour stromal proteomes.

It is an object of the invention to provide methods for treating breast cancer, methods of obtaining indications of the prognosis of breast cancer subjects, for classifying breast tumours and for predicting the therapeutic effectiveness of radiotherapy treatment on a subject with breast cancer.

In one embodiment, the invention provides a method of obtaining an indication of the prognosis of breast cancer in a subject, the method comprising the step:
(a) producing a signature score from normalised levels of at least 3 biomarkers, wherein the at least 3 biomarkers are selected from a first group consisting of GAPDH, HSPA4, LDHA and VASP
   and wherein the selected biomarkers were obtained from a biological sample which was obtained from the subject;
wherein the produced signature score is indicative of the prognosis of breast cancer in the subject.

In another embodiment, the invention provides a method of classifying breast tumours, the method comprising the steps:
(a) producing a signature score from normalised levels of at least 3 biomarkers, wherein the at least 3 biomarkers are selected from a first group consisting of GAPDH, HSPA4, LDHA and VASP,
   and wherein the selected biomarkers were obtained from a biological sample which was obtained from the subject; and
(b) classifying the breast tumour based on the signature score produced.

In a further embodiment, the invention provides a method of predicting the therapeutic efficacy of radiotherapy treatment on a subject with breast cancer, the method comprising the step:
(a) producing a signature score from normalised levels of at least 3 biomarkers, wherein the at least 3 biomarkers are selected from a first group consisting of GAPDH, HSPA4, LDHA and VASP,
   and wherein the selected biomarkers were obtained from a biological sample which was obtained from the subject;
wherein the produced signature score is predictive of the therapeutic efficacy of radiotherapy treatment on the breast cancer.

In some embodiments of the invention, the method is carried out in vitro or ex vivo.

As demonstrated herein, the signature score (e.g. 33P) is associated with large tumour size, high histologic grade, lymph node metastases, ER negative tumour and a basal-like phenotype.

In one embodiment, therefore, the invention provides a method of obtaining an indication of the prognosis of breast cancer in a subject, the method comprising the step:
(a) producing a signature score from normalised levels of at least 3 biomarkers, wherein the at least 3 biomarkers are selected from a first group consisting of: GAPDH, HSPA4, LDHA and VASP, and wherein selected biomarkers were obtained from a biological sample which was obtained from the subject;
wherein the produced signature score is indicative of the prognosis of breast cancer in the subject.

The subject is preferably a human subject. The human may, for example, be 0-10, 10-20, 20-30, 30-40, 40-50, 50-60, 60-70, 70-80, 80-90, 90-100 or above 100 years old. The human may be one who is suffering from or at risk from a particular disease or disorder, e.g. cancer, preferably breast cancer. In some preferred embodiments, the subject is one who is suffering from or who has previously suffered from cancer, e.g. breast cancer. In some embodiments, the subject is one who has previously been treated for breast cancer, e.g. by surgery and/or chemotherapy and/or radiotherapy. A control subject may be defined as a non-diseased subject, a subject without breast cancer, a typically-developed subject or a healthy-aged subject.

In some embodiments, the biomarkers are selected from a first group (referred to herein as “4P”) consisting of: GAPDH, HSPA4, LDHA and VASP. These 4 biomarkers were found to have a significant association value in a METABRIC analysis. At least 3 biomarkers were selected from this first group. In some embodiments, all 4 biomarkers were selected from this first group.

In some embodiments, the biomarkers are selected from a second group (referred to herein as “9P”) consisting of COL5A1, GAPDH, HNRNPF, HSPA4, IDH1, LDHA, PGK1, SET and VASP. This second group includes all of the first group of biomarkers. These biomarkers represent the overlap between: (a) the 18 proteins (“18P”) which were found using a reduction algorithm on the 33P proteins; and (b) the 13 proteins (“13P”) which were found to be significantly associated with hypoxia.

At least 5 biomarkers were selected from this second group. In some embodiments, at least 6, 7, 8 or 9 biomarkers were selected from this second group. Preferably, at least 7 biomarkers were selected from this second group. In some embodiments, all 9 biomarkers were selected from this second group.

In some embodiments, the biomarkers were selected from a third group (referred to herein as “13P”) consisting of COL5A1, RNPEP, AK2, GAPDH, GSTO1, HNRNPF, HSPA4, IDH1, LDHA, NPM1, PGK1, SET and VASP. This third group includes all of the first and second group of biomarkers. At least 5 biomarkers were selected from this third group. In some embodiments, at least 6, 7, 8, 9, 10, 11, 12 or 13 biomarkers were selected from this third group. Preferably, at least 10 biomarkers were selected from this third group. In some embodiments, all 13 biomarkers were selected from this third group.

In some embodiments, the biomarkers were selected from a fourth group (referred to herein as “18P”) consisting of CDC37, COL5A1, CTSB, GAPDH, GRB2, HNRNPA1, HNRNPD, HNRNPF, HSPA4, HSPA9, IDH1, LDHA, MYL6, P4HB, PGK1, RRBP1, SET and VASP. This fourth group includes all of the markers from the first, second and third groups. At least 5 biomarkers were selected from this fourth group. In some embodiments, at least 6, 7, 8, 9, 10, 11, 12, 13, 14, 15, 16, 17 or 18 biomarkers were selected from this fourth group. Preferably, at least 15 biomarkers were selected from this fourth group. In some embodiments, all 18 biomarkers were selected from this fourth group.

In some embodiments, the biomarkers were selected from a fifth group (referred to herein as “33P”) consisting of ACY1, COL5A1, RNPEP, ABRACL, AK2, CALU, CDC37, CNDP2, CNPY2, COPE, COX6B1, CTSB, GAPDH, GRB2, GSTO1, HNRNPA1, HNRNPD, HNRNPF, HSPA4, HSPA9, IDH1, IDH2, LDHA, MDH2, MYL6, NPM1, P4HB, PGK1, RCN1, RRBP1, S100A4, SET and VASP. This fourth group includes all of the markers of the first, second, third and fourth groups. At least 10 biomarkers were selected from this fifth group. In some embodiments, at least 11, 12, 13, 14, 15, 16, 17, 18, 19, 20, 21, 22, 23, 24, 25, 26, 27, 28, 29, 30, 31, 32 or 33 biomarkers were selected from this fourth group. Preferably, at least 20 or 25 biomarkers were selected from this fifth group. In some embodiments, all 33 biomarkers were selected from this fifth group.

The biomarkers may be protein biomarkers or RNA (preferably mRNA) biomarkers. The corresponding protein identifiers of the biomarker genes are given in Supplementary Table 3. References to these biomarkers (whether protein or RNA) include references to naturally-occurring human variants of these biomarkers. In some embodiments of the invention, the method includes the step of selecting the biomarkers.

In some embodiments, the biological sample is a sample of blood, serum or plasma. Serum and plasma may be obtained from a blood sample from the subject, wherein the blood cells have been removed. In such cases, the biomarkers are proteins.

In other embodiments, the biological sample is a whole tissue breast tumour sample. This contains a mixture of tumour cells and tumour stroma. In some preferred embodiments, the biological sample is obtained by needle biopsy of the breast cancer or from a surgical specimen (resection) from the breast cancer. In such cases, the biomarkers are proteins or mRNA, preferably mRNA.

The biological sample is a sample which is obtained or which has previously been obtained from the subject. In some embodiments, the method additionally comprises the step of obtaining one or more biological samples from the subject.

The term “tumour stroma” relates to the non-malignant cells and the extracellular matrix which are present in the tumour microenvironment. The stroma comprises a variable portion of the entire tumour: up to 90% of a tumour may be stroma, with the remaining 10% as cancer cells. Many types of cells are present in the stroma, but four abundant types are fibroblasts, T cells, macrophages and endothelial cells.

In some embodiments, the biomarkers are either all proteins or all RNA. In other embodiments, the biomarkers are a mixture of protein and RNA. Preferably, the biomarkers are either all protein or all RNA.

In some embodiments, the biomarkers are proteins. The proteins may be obtained from the biological sample by any suitable method. The protein biomarkers may, for example, be identified by MS analysis or shotgun proteomics analysis.

In other embodiments, the biomarkers are RNA, preferably mRNA. RNA may be extracted by any suitable method. The RNA biomarkers may, for example, be identified by RNASeq or qRT-PCR.

The signature score is a numerical value which is representative of the overall (normalised) expression levels (either protein expression levels or (m) RNA expression levels) of the selected biomarkers in the biological sample. Normalisation of each of the biomarker levels is necessary in order to obtain an accurate interpretation of the signature score. The normalisation step may be performed using any suitable method. Numerous such methods are known in the art.

In some embodiments, the levels of each selected biomarkers are normalised against the levels of one or more control or housekeeping genes/proteins which are expressed in the selected biological sample, preferably control or housekeeping genes/proteins which have low variability in their expression levels in the selected biological sample (e.g. in blood or in breast cancer tissue).

The levels of the one or more control or housekeeping genes/proteins are levels which are or have been obtained from the selected biological sample (i.e. the actual same sample from which levels of the biomarkers are obtained).

For example, Tilli et al. (BMC Genomics (2016)17:639) identified a set of control genes including CCSER2, SYMPK, ANKRD17 and PUM1. These were found to be usable in the clinical analyses of breast cell lines and tissue samples. The levels of each of the biomarkers used in the invention may be normalised against the levels of one or more of the latter control genes in the biological sample.

In another example, the Oncotype DX 21-gene test uses 5 housekeeping genes to normalize their 16 cancer-related genes. These 5 housekeeping genes are ACTB, GAPDH, GUS, RPLP0 and TFRC. The levels of each of the biomarkers used in the invention may be normalised against the levels of one or more of the latter control genes/proteins in the biological sample.

In another example, the Prosigna® Breast Cancer Prognostic Gene Signature Assay uses 8 housekeeping genes to normalize their 50 (PAM50) cancer-related genes. These 8 housekeeping genes are ACTB, MRPL19, PUM1, SF3A1, GUSB, PSMC4, RPLP0 and TFRC. The levels of each of the biomarkers used in the invention may be normalised against the levels of one or more of these control genes/proteins in the biological sample.

In some embodiments, the normalisation step involves subtracting the level of one or more control or housekeeping genes or proteins from the obtained level of each selected biomarker. In other embodiments, the normalisation step involves dividing the obtained level of each selected biomarker by the level of one or more control or housekeeping genes or proteins.

The signature score is then produced by summing the normalised levels of each of the selected biomarkers.

A weighting may be added to or multiplied to one or more of the normalised biomarker levels before those levels are summed (e.g. (k1×BM1)+(k2×BM2)+ . . . , where k1 and k2 are independently numbers which may be the same or different, and BM1 and BM2 are the determined levels of two of the biomarkers).

In embodiments of the invention wherein more than one signature scores are compared, the signature scores are all produced in the same way.

In embodiments of the invention which refer to “corresponding biomarkers”, this is referring to the same biomarkers as the previously-mentioned biomarkers. For example, if the first signature score is produced using 5 of the 9P biomarkers, then the second signature score is also produced using the same 5 9P biomarkers.

In embodiments of the invention which refer to “corresponding biological samples”, this is referring to the same biological samples as the previously-mentioned biological samples. For example, if the first signature score is produced from mRNA, then the second signature score is also produced using mRNA.

As used herein, the term “reference signature score” or “corresponding reference signature score” refers to a signature score which has been produced using the same (i.e. corresponding) parameters as the signature score to which it is being compared, (e.g. the same type of biomarkers (e.g. protein or RNA), the same number of biomarkers (e.g. 33), the same set of biomarkers (e.g. 33P) from the same type of biological sample (e.g. serum) and using the same normalisation steps) wherein the biomarkers for the reference signature score were obtained from control (e.g. healthy) subjects (and not from the subject with breast cancer). Thus the reference signature score provides a baseline from a control subject against which to compare the subject with breast cancer's signature score.

In some embodiments of the invention, the signature score is indicative of the prognosis of breast cancer in the subject. A comparison of the signature score from the subject to that of a corresponding reference signature score provides an indication of the prognosis of breast cancer in the subject, the likely outcome or course of the breast cancer in the subject or the chance of recovery of the subject.

As used herein, the term “is indicative of the prognosis of breast cancer in the subject” means that there is a negative correlation between the signature score and a good prognosis of breast cancer in that subject.

Consequently, a signature score from the subject which is higher than a corresponding reference signature score (e.g. from a healthy control subject or a control subject without breast cancer) means an increased likelihood or statistically-significant chance (where the difference is significant) of the subject having a poor prognosis for breast cancer. The reference signature score may also be one which has been obtained from a subject having breast cancer but with a good prognosis. The reference signature score may also be one which has been obtained from a cohort of subjects having low-grade breast cancers.

In this case, the extent of the difference between the signature score from the subject and the reference signature score (e.g. from a healthy control subject or a control subject without breast cancer) provides an indication of the degree of the poor prognosis of the subject.

Furthermore, a signature score from the subject which is lower than a corresponding reference signature score (e.g. from a breast tumour sample from a breast cancer subject) means an increased likelihood or statistically-significant chance (where the difference is significant) of the subject having a good prognosis for breast cancer.

The method may comprise the additional step of administering a treatment appropriate for treating the breast cancer to the subject if the produced signature score is indicative of the subject having a poor prognosis for breast cancer.

In another embodiment, the invention provides a method of classifying breast tumours, the method comprising the steps:
(a) producing a signature score from the normalised levels of at least 3 biomarkers, wherein the at least 3 biomarkers are selected from a first group consisting of GAPDH, HSPA4, LDHA and VASP, and wherein the selected biomarkers were obtained from a biological sample which was obtained from the subject; and
(b) classifying the breast tumour based on the signature score obtained.

The biomarkers are selected and the signature score is produced as disclosed herein. The biological sample is one as disclosed herein. The levels of the selected biomarkers are normalised as disclosed herein.

In this embodiment of the invention, the obtained signature score is compared against a corresponding panel of reference signature scores or set of ranges of references signature scores which have (previously) been obtained from tissues which are representative of different breast tumours having different phenotypes or genotypes or other physical properties; and classifying the breast tumour based on which reference signature score is closest to the obtained signature score, or into which range of reference scores the obtained signature score falls.

In some embodiments, the term “classifying the breast tumour based on the signature score obtained” refers to classifying the tumour on the basis of breast tumour type.

The breast tumour may be any type of breast tumour, e.g. Luminal A (LumA), Luminal B (LumB), Basal-like (Basal), HER2-enriched (HER2) breast tumour or Normal-like. Preferably, the breast tumour is a Luminal A breast tumour.

In this embodiment, the obtained signature score is compared against a corresponding panel of reference signature scores which have (previously) been obtained from tissues which are representative of different breast tumour types; and classifying the tumour based on which reference signature score is closest to the obtained signature score.

In other embodiments, the term “classifying the breast tumour based on the signature score obtained” refers to classifying the tumour on the basis of the tumour's size.

In this embodiment, the obtained signature score is compared against a corresponding panel of reference signature scores which have (previously) been obtained from tissues which are representative of different breast tumour sizes; and classifying the tumour based on which reference signature score is closest to the obtained signature score.

In other embodiments, the term “classifying the breast tumour based on the signature score obtained” refers to classifying the tumour on the basis of histologic grade.

In this embodiment, the obtained signature score is compared against a corresponding panel of reference signature scores which have (previously) been obtained from tissues which are representative of different breast tumour histologic grades; and classifying the tumour based on which reference signature score is closest to the obtained signature score.

In other embodiments, the term “classifying the breast tumour based on the signature score obtained” refers to classifying the tumour on the basis of its likelihood of having lymph node metastases.

In this embodiment, the obtained signature score is compared against a corresponding panel of reference signature scores which have (previously) been obtained from tissues which are representative of breast tumours having lymph node metastases or not; and classifying the tumour based on which reference signature score is closest to the obtained signature score.

In other embodiments, the term “classifying the breast tumour based on the signature score obtained” refers to classifying the tumour as being ER negative or not.

In this embodiment, the obtained signature score is compared against a corresponding panel of reference signature scores which have (previously) been obtained from tissues which are representative of breast tumours which are ER negative or not; and classifying the tumour based on which reference signature score is closest to the obtained signature score.

In other embodiments, the term “classifying the breast tumour based on the signature score obtained” refers to classifying the breast tumour as having a basal-like phenotype or not. In this embodiment, the obtained signature score is compared against a corresponding panel of reference signature scores which have (previously) been obtained from tissues which are representative of breast tumours having a basal-like phenotype or not; and classifying the tumour based on which reference signature score is closest to the obtained signature score.

In other embodiments, the term “classifying the breast tumour based on the signature score obtained” refers to classifying the tumour on the basis of as having high levels of tumour cell proliferation. In this embodiment, the obtained signature score is compared against a corresponding panel of reference signature scores which have (previously) been obtained from tissues which are representative of different breast tumours having high levels of tumour cell proliferation or not; and classifying the tumour based on which reference signature score is closest to the obtained signature score.

In yet other embodiments, the invention provides a method of predicting the therapeutic efficacy of radiotherapy treatment on a subject with breast cancer, the method comprising the step:
(a) producing a signature score from normalised levels of at least 3 biomarkers, wherein the at least 3 biomarkers are selected from a first group consisting of GAPDH, HSPA4, LDHA and VASP, and wherein biomarkers were obtained from a biological sample which was obtained from the subject;
wherein the signature score is predictive of the therapeutic efficacy of radiotherapy treatment on the breast cancer.

The biomarkers are selected and the signature score is produced as disclosed herein. The biological sample is one as disclosed herein. The levels of the selected biomarkers are normalised as disclosed herein. As used herein, the term “is predictive of the therapeutic efficacy of radiotherapy treatment on the breast cancer” means that there is a negative correlation between the signature score and a longer survival time of the subject after radiotherapy treatment for the breast cancer.

In this embodiment of the invention, the obtained signature score is compared against a corresponding reference signature score which has (previously) been obtained from a healthy control subject or a control subject without breast cancer.

Consequently, a signature score from the subject which is higher than a corresponding reference signature score (e.g. from a healthy control subject or a control subject without breast cancer) means an increased likelihood or statistically-significant chance (where the difference is significant) of the subject having a shorter survival time after radiotherapy treatment. This provides an indication that radiotherapy treatment of the subject is unlikely to be efficacious.

Furthermore, a signature score from the subject which is lower than a corresponding reference signature score (e.g. from a breast tumour sample from a breast cancer subject) means an increased likelihood or statistically-significant chance (where the difference is significant) of the subject having a longer survival time after radiotherapy treatment.

Based on the teachings of the invention, it should also be possible to define a threshold value below which radiotherapy treatment is recommended for the subject, and above which radiotherapy treatment is not recommended for the subject.

In another embodiment, the invention provides a method of obtaining an indication of the efficacy of a drug which is being used to treat breast cancer in a subject, the method comprising the steps:
(a) producing a first signature score from normalised levels of 3 or more biomarkers, wherein the at least 3 biomarkers are selected from a first group consisting of GAPDH, HSPA4, LDHA and VASP, and wherein the at least 3 biomarkers were obtained from a first biological sample which was obtained from the subject at a first time point; and
(b) producing a second signature score using corresponding levels of the corresponding biomarkers in a corresponding second biological sample obtained from the subject at a second (later) time point;
wherein the drug has been administered to the subject in the interval between the first and second time points, wherein a decrease in the second signature score compared to the first signature score is indicative of the efficacy of the drug, and wherein an increase in the second signature score compared to the first signature score is indicative of the lack of efficacy of the drug.

In all methods of the invention, the increase and/or the decrease is preferably a significant one. Significance may be measured, for example, using Student's t-test, with a p-value significance threshold set to 0.05.

In another embodiment, the invention provides a method of treating breast cancer in a subject, the method comprising the steps of:
(a) producing a signature score from normalised levels of at least 3 biomarkers, wherein the at least 3 biomarkers are selected from the group consisting of GAPDH, HSPA4, LDHA and VASP, and wherein the at least 3 biomarkers were obtained from a biological sample which was obtained from the subject;
(b) comparing the signature score with a reference signature score (e.g. one from a healthy control subject or from a subject without breast cancer); and
(c) administering a treatment appropriate for treating breast cancer to the subject if the signature score is above the reference signature score, thereby treating the breast cancer in the subject.

In another embodiment, the invention provides a method of treating breast cancer in a subject, the method comprising the steps of:
(a) administering a treatment appropriate for treating breast cancer to the subject, wherein, prior to administration, a signature score which was produced from normalised levels of at least 3 biomarkers, wherein the at least 3 biomarkers are selected from the group consisting of GAPDH, HSPA4, LDHA and VASP, and wherein the at least 3 biomarkers were obtained from a biological sample which was obtained from the subject,
had been determined to be above a reference signature score (e.g. one from a healthy control subject or from a subject without breast cancer);
In yet another embodiment, the invention provides a method of treating breast cancer in a subject, the method comprising the steps of:
(a) receiving a signature score which was produced from normalised levels of at least 3 biomarkers, wherein the at least 3 biomarkers were selected from the group consisting of GAPDH, HSPA4, LDHA, and VASP, and wherein the at least 3 biomarkers were obtained from a biological sample which was obtained from the subject; and
(b) identifying the subject as having a signature score above a reference signature score (e.g. one from a healthy control subject or from a subject without breast cancer),
thereby providing an indication of the potential efficacy of administering treatment appropriate for treating the breast cancer to the subject, and administering treatment appropriate for treating the breast cancer to the subject.

In all embodiments of the invention, the method may comprise the additional step of administering a treatment appropriate for treating the breast cancer to the subject.

Treatments for breast cancer are well known in the art, including treatment with surgery, which may be followed by chemotherapy or radiation therapy, or both. For example, the following list includes some of the commonly used adjuvant chemotherapy for breast cancer:
CMF: cyclophosphamide, methotrexate, and 5-fluorouracil.
FAC (or CAF): 5-fluorouracil, doxorubicin, cyclophosphamide.
AC (or CA): Adriamycin (doxorubicin) and cyclophosphamide.
AC-Taxol: AC followed by paclitaxel (Taxol).
TAC: Taxotere (docetaxel), Adriamycin (doxorubicin), and cyclophosphamide.
FEC: 5-fluorouracil, epirubicin and cyclophosphamide.
AT Adriamycin (doxorubicin) and Taxotere (docetaxel).

In yet another embodiment, the invention provides a method of screening for agents for treating breast cancer, the method comprising the steps:
(a) producing a first signature score from normalised levels of at least 3 biomarkers, wherein the at least 3 biomarkers are selected from the group consisting of GAPDH, HSPA4, LDHA and VASP, and wherein the at least 3 biomarkers were obtained from a breast cancer sample which has been treated with an agent;
(b) producing a second signature score from corresponding biomarkers obtained from the breast cancer sample which has not been treated with the agent; and
(c) comparing the first and second signature scores;
wherein a decrease in the first signature score compared to the second signature score is indicative of an agent which is capable of treating breast cancer.

The breast cancer sample may be a breast cancer cell line, e.g. MCF-7 or MDA-MB-231; or a sample (e.g. tissues or cells) of a breast cancer from a subject (which may be used in the form of a cell line, spheroid or organoid).

Agents which are identified as being capable of treating breast cancer on the basis of samples of breast cancer from a subject may then be formulated for administration to the subject, and then optionally administered to the subject.

In yet another embodiment, the invention provides a method of predicting the risk of recurrence of breast cancer in a subject who has previously had breast cancer but who is currently in remission, the method comprising the step:
(a) producing a signature score from normalised levels of at least 3 biomarkers, wherein the at least 3 biomarkers are selected from the group consisting of GAPDH, HSPA4, LDHA and VASP, and wherein biomarkers were obtained from a biological sample which was obtained from the subject;
wherein the signature score is predictive of the risk of recurrence of breast cancer in the subject.

The biomarkers are selected and the signature score is produced as disclosed herein. The biological sample is one as disclosed herein. The levels of the selected biomarkers are normalised as disclosed herein.

As used herein, the term “is predictive of the risk of recurrence of breast cancer in the subject” means that there is a positive correlation between the signature score and the risk of recurrence of breast cancer in the subject. In particular, a signature score from the subject which is higher than a corresponding reference signature score (e.g. from a healthy control subject or a control subject without breast cancer) means an increased likelihood or statistically-significant chance (where the difference is significant) of recurrence of breast cancer in the subject. In particular, a signature score from the subject which is lower than a corresponding reference signature score (e.g. from a breast tumour sample from a breast cancer subject) means a decreased likelihood or statistically-significant chance (where the difference is significant) of recurrence of breast cancer in the subject.

In yet another embodiment, the invention provides a kit comprising reagents sufficient for the detection and/or quantitation of at least 3 of the following biomarker genes: GAPDH, HSPA4, LDHA and VASP, characterised in that said reagents comprise a plurality of forward and reverse primers pairs, wherein said forward and reverse primers pairs are selected from forward and reverse primer pairs which are capable of identifying at least 3 of the following genes: GAPDH, HSPA4, LDHA and VASP.

In yet a further embodiment, the invention provides a method of detecting biomarkers in a breast tissue sample obtained from a human subject, the method comprising measuring:
(i) a protein expression level for every protein in a group of classifier proteins; or
(ii) a mRNA expression level for every gene in a group of classifier genes;
wherein the group of classifier proteins or classifier genes consists of only:
(i) 4P; (ii) 9P; (iii) 13P; (iv) 18P; or (v) 33P, wherein 4P, 9P, 13P 18P and 33P are the groups of proteins or groups of genes as defined herein.

Preferably, the method steps are carried out (one after the other) in the order specified.

The disclosure of each reference set forth herein is specifically incorporated herein by reference in its entirety.

### BRIEF DESCRIPTION OF THE FIGURES

FIG. 1: Schematic overview of study with laboratory and data analysis.

Methods workflow of proteomics experiments of breast cancer cell line (BCCL) conditioned media (a) and formalin-fixed paraffin-embedded (FFPE) tumor samples (b). From hypoxic BCCL secretome experiments, 150 proteins showed hypoxia-increased secretion (Hx). From microdissected FFPE material, 283 proteins showed subtype differences only in the stromal compartment (basal-like (BL) vs. luminal-like (LL) subtypes). The 33-protein hypoxia stromal signature (33P) was generated from the overlapping proteins between the 150 hypoxia-increased proteins (BCCL secretome experiments) and the 283 proteins showing stroma-exclusive subtype differences (microdissected breast cancer patient material) (c). The hypoxia response proteins and 33P signature were validated using bioinformatic analysis and experimental validation (d). The hypoxia response proteins were investigated with bioinformatic analyses (gene ontology analysis (GO), gene set enrichment analysis (GSEA), ingenuity pathway analysis (IPA), network biology analysis using Cytoscape). The 33P signature was explored bioinformatically (GSEA, connectivity map analysis (CMAP), Cibersort, Search-Based Exploration of Expression Compendium (SEEK)) and validated with external clinical validation (METABRIC-Discovery, n=852; KMplotter merged cohorts) for survival analysis and permutation test, and with extended cell line validation (BCCLs; LL n=6, BL n=6).

33P: 33-protein hypoxia stromal signature. BCCL: breast cancer cell line. BL: basal-like breast cancer subtype. CMAP: Connectivity map analysis. ELISA: enzyme-linked immunosorbent assay. FFPE: formalin-fixed paraffin-embedded tissue. GO: gene ontology analysis. GSEA: gene set enrichment analysis. Hx: hypoxia. IHC: immunohistochemistry. LL: luminal-like breast cancer subtype. MS: mass spectrometry. Nx: normoxia. SEEK: search-based exploration of expression compendium.

FIG. 2: Comparing hypoxic and normoxic secretomes in luminal-like and basal-like breast cancer cell lines.

Gene set enrichment analysis of secretome data ranked using a two-sided t-test from hypoxia-increased (blue) to hypoxia-decreased (green) in luminal-like (a, c, e, g) and basal-like (b, d, f, h) cell lines. The selected analyses show significant enrichment of KEGG pathways glycolysis, TCA cycle, oxidative phosphorylation and angiogenesis (GOID 1525) in the luminal-like hypoxic secretome. The basal-like hypoxic secretome was not enriched in either of the gene sets. Ranking the secretome data from basal-like (red) to luminal-like (blue) under normoxic (i) and hypoxic (j) conditions, showed an enrichment in angiogenic proteins in the basal-like subtype in both oxygen conditions. P-values were not adjusted for multiple testing. GSEA: gene set enrichment analysis. RES: running enrichment score. KEGG: Kyoto Encyclopedia of Genes and Genomes. GO: gene ontology analysis.

FIG. 3: Survival plots in breast cancer patients scored by 33P hypoxia stromal signature (METABRIC-Discovery cohort)

Kaplan-Meier of breast cancer specific survival in patients diagnosed with luminal-like and basal-like breast cancer (n=852) (a), only luminal-like subtype (n=734) (b), and only basal-like subtype (n=118) (c) in the METABRIC-Discovery cohort. The patients are divided into quartiles depending on 33P signature score (33P-low, Q1 in blue; 33P-high, Q4 in red). The plots show a significant association between high 33P scores (Q4) and poor survival for patients diagnosed with luminal-like and basal-like breast cancer. Survival differences between groups were evaluated with a two-sided log-rank test.

FIG. 4: Interaction between the 33P signature and radiotherapy (METABRIC-Discovery cohort)

Univariate survival analysis (Kaplan-Meier method) of breast cancer specific survival in BC patients from the METABRIC-Discovery cohort. The patients were grouped into four 33P score quartiles (33P-low, Q1 in blue—33P-high, Q4 in red). The top panels (a, b) include all patients (luminal-like and basal-like; n=852), the bottom include only Luminal A patients (n=466) (c, d). The patients were stratified into patients that did not (a, c) or did (b, d) receive radiotherapy. Survival differences between groups were evaluated with a two-sided log-rank test. The Y-axes of FIGS. 4a-4d all refer to the Probability of Survival, with the same scales.

FIG. 5: Permutation test.

Histogram of cumulative chi-square statistics values after 10,000 permutations. In each permutation, 33 proteins were selected at random from a pool of the 150 hypoxia-increased and 283 stroma proteins from which the 33P was derived, and the one-sided chi-square statistics from a univariate survival analysis (Kaplan-Meier method) were extracted. The dotted red line shows the chi-square statistics of the 33P signature. The p-value is calculated from the proportion of permutations that give a higher Chi-Square value divided by the total number of permutations.

FIG. 6: Signature correlations.

33P hypoxia stromal signature correlates with signatures for tissue hypoxia (a-c), proliferation (d-e), glycolysis (f-h), vascular proliferation (i-j), and signatures reflecting EMT (k) and stemness (I-m), a luminal progenitor signature (n) and correlates negatively with mature luminal signature (o) in the METABRIC-Discovery mRNA cohort. ρ: Spearman's rank correlation coefficient. p: Spearman's rho test (two-sided).

FIG. 7: Survival plots in breast cancer patients scored by 33P hypoxia stromal signature.

Kaplan-Meier plots of luminal A (n=466) (a) and luminal B (n=268) (b) from the METABRIC-Discovery mRNA cohort. The plots show a significant association between high signature scores and poor survival for luminal-like A subtype. Validation in the merged cohorts from KMplotter (updated n=4934) (c), and also stratified for luminal A (d), luminal B (e), and basal-like subtype (f), show significantly lower probability of survival of patients with high 33P scores. Red lines represent the 33P high (upper quartile, Q4) group, and the blue line represents the rest (Q1-Q3). Survival differences between groups were evaluated with a two-sided log-rank test.

FIG. 8: Reduction analysis of 33P identifies a ‘peak signature’ of 18 proteins.

The 33P signature was reduced by recursively leaving one gene out of the signature and then testing the predictive strength of the remaining N−1 genes in a survival analysis (Q1-3 vs Q4, METABRIC-Discovery cohort, n=852). The strongest N−1 signature (lowest Log-rank p-value) was retained, and the process was repeated until only one gene remained. (a) The mountain-like plot shows the log-rank p-value for each iteration. The red line represents the “peak-signature”, i.e., the reduced version of 33P (18-proteins) that showed the largest effect on survival (p=4.3×10⁻¹⁷, compared to baseline 33P p=1.0 10-8). The 18-proteins were CDC37, COL5A1, CTSB, GAPDH, GRB2, HNRNPA1, HNRNPD, HNRNPF, HSPA4, HSPA9, IDH1, LDHA, MYL6, P4HB, PGK1, RRBP1, SET and VASP. (b-e) Univariate survival analysis (Kaplan-Meier method) of the 18-protein “peak”-signature in all patients (n=852), Luminal A (n=466), Luminal B (n=268) and basal-like (n=118) in the METABRIC-Discovery cohort. The reduction analysis was based on “all patients”. Still, after stratifying the patients we observed a lower survival in Q4 of both the luminal A and luminal B subtypes compared to the original 33P-signature. Red lines represent the 33P high (upper quartile, Q4) group, and the blue line represents the rest (Q1-Q3). Survival differences between groups were evaluated with a two-sided log-rank test.

FIG. 9: Protein-protein association network of the 33P proteins.

The protein-protein association network shows that 29 of the 33 proteins are connected to one large network. Thickness of lines between proteins represents strength of association. The blue colored nodes are associated with “VEGFA-VEGFR2 signaling pathway” (WikiPathways; WP3888; p<0.001). The figure was generated from string-db.org.

FIG. 10: Immunohistochemical staining of NRF2 in breast cancer tissues.

Evaluation of NRF2 expression in breast cancer tissue microarrays by immunohistochemistry (n=42; ×400 magnification, scale bar 50 μm), showing weak (a) and moderate (b) stromal staining. Stromal expression is indicated with black arrows, and tumor epithelial staining is indicated with white arrows. Stronger stromal expression of NRF2 is positively correlated with 33P scores (MS-proteomics) in the same samples (p=0.05), but tumor cell expression is not associated with 33P.

FIG. 11: Scatterplot of the 33P discovery signature score against the 36P validation signature score.

Each dot represents one patient in the METABRIC-Discovery cohort (n=852; luminal-like and basal-like only). A Pearson correlation coefficient (r) of 0.70 suggests a strong correlation between the discovery and validation signatures. Statistical test: two-tailed t-test.

FIG. 12: Gene set enrichment analysis of 33P in the hypoxia validation dataset.

The identified proteins in the validation dataset were ranked by p-value (all samples, paired t-test (two-sided), no adjustment was performed since only one gene set was tested—hypoxia vs. normoxia) and tested against the 33P proteins in a gene set enrichment analysis. The analysis showed a significant enrichment of 33P in the hypoxia validation dataset (p=0.02; NES 1.45). The figure was generated using the fgsea R-package. ES: enrichment score; NES: normalized enrichment score.

FIG. 13: Univariate survival analysis (Kaplan-Meier method) of patients from METABRIC-Discovery cohort and KMplotter by expression of the 13P genes.

Patients in the METABRIC-Discovery cohort were grouped into four quartiles (Q1-Q4) based on the expression of the 13P genes, and both (a) all patients and (b) the patients diagnosed with luminal A breast cancer showed worse probability of survival in the high 13P group. These data were supported by KMplotter, where high 13P (upper quartile) was associated with worse survival in (c) all patients (n=2032), (d) luminal A (n=633), (e) luminal B (n=466) and (f) basal-like patients (n=442). Survival differences between groups were evaluated with a two-sided log-rank test.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The following Materials and Methods were used in one or more of the Examples.

### Selection of Breast Cancer Cell Lines.

Selection of breast cancer cell lines (BCCL) for the discovery phase (4 BCCLs; luminal-like n=2, basal-like n=2) and the extended validation experiments (8 additional BCCLs; luminal-like n=4, basal-like n=4) was based on literature studies and bioinformatic mapping^{59,60} By combining mapping of existing literature information with in-house bioinformatics analyses (below), we provide stronger evidence on the molecular suitability of candidate cell lines, for the selection of cell lines and for extended validation experiments. This information is summarized in Supplementary Table 1. The initially selected luminal-like cell lines are both ER and PR positive, and both selected basal-like cell lines are triple-negative. These cell lines were selected with a balance between primary and metastatic source (Supplementary Table 1). The selected cell lines are widely used and included in several large studies investigating breast cancer cells in vitro⁶¹⁻⁶⁶. All selected cell lines are part of at least one of American Type Culture Collection (ATCC)'s cell line panels for breast cancer or triple-negative breast cancers, and none of the included cell lines are among the cell lines with debated subtype or characteristics (e.g., SKBR3, previously classified as luminal^{61,63}, and later classified as HER2-enriched⁶²).

To identify representative cell lines for the validation panel, we performed an unbiased exploratory analysis using publicly available transcriptomic (n=54) and proteomic (n=28) data from the Cancer Cell Line Encyclopedia (CCLE)^{59,60}. For both transcriptomic and proteomic datasets, we used the available gene expression and protein expression matrices as input. The cell lines were projected into the 2D space using multidimensional scaling (MDS).

The cell lines formed clusters, and the clusters were strongly driven by their molecular subtype identity. This information was used as a guide to assess differences in the expression profiles of the available cell lines (n=4), and unbiasedly select new candidate cell lines to cover the observed 2D space (validation cell lines, n=8). We believe that the original four cell lines were neither outliers nor expressing very different transcriptomic or proteomic profiles from all other cell lines. Instead, they were quite representative in the 2D subtype space, as were the additional 8 cell lines that we subsequently selected. Expanding the cell line panel of luminal-like cell lines: we decided to include a HER2-positive cell line consistent with the luminal B tumor subtype, and three cell lines with hormone receptor status patterns corresponding to luminal A tumors. Importantly, regarding the HER2-positive cell lines included in our study (initial: BT-474; additional: ZR-75-30); these cell lines are hormone receptor positive and have luminal characteristics, and belong to the luminal category of cell lines.

Expanding the cell line panel of basal-like cell lines: three basal A cell lines and one basal B and claudin-low cell line were included to also have a balance between basal A and basal B cell lines in follow-up experiments. Importantly, all six basal-like cell lines were triple-negative. The basal A cell lines were included as this category is corresponding closely with the basal-like tumor subtype^{9,67}, and the basal B category of cell lines were included since these are more similar to the triple-negative tumors. When selecting cell lines for the validation experiment, we carefully selected cell lines with similar media and supplements to ensure that there was no obvious external metabolic bias between the luminal and basal-like subtypes.

All cell lines were provided from American Type Culture Collection (ATCC) with certificate of analysis. All cell lines tested negative for mycoplasma contamination.

### Selection of Patients and Study Approval.

For the in-house human tumor samples used in our study (for microdissection and proteomics, n=24; for immunohistochemistry, n=42; see below), the protocol was approved by the Western Regional Committee for Medical and Health Research Ethics, REC West (REK #2014/1984). The informed consent was waived by the REC West Committee, based on national guidelines, as well as the age and size of the full cohort covered by the approval. However, the actual patients included were informed about the research project and the possibility to withdraw. All studies were performed in accordance with guidelines and regulations by the University of Bergen and REK, and in accordance with the Declaration of Helsinki Principles.

Tumor tissues (n=24) were collected from female patients (aged 50-69 years) diagnosed with breast carcinoma NST (no special type) during 1996-2003, as part of a prospective and population-based screening program. Sex was defined by the national and unique 11-digit personal identification number. Tissue sections from 24 primary tumors (12 basal-like, 6 luminal A, 6 luminal B) were included for microdissection; tumor categories were based on the St Gallen 2013 classification⁶⁸. All basal-like samples were also triple-negative, and all luminal samples were estrogen and progesterone positive, and HER2-negative. The luminal B tumors displayed more than 15% Ki67-positive nuclei⁶⁹

### Cell Cultures.

For the discovery experiments, BT-474 (ATCC® HTB-20™) was grown in RPMI medium, MCF7 (ATCC® HTB-22™) and Hs 578T (ATCC® HTB-126™) were grown in DMEM medium and MDA-MB-231 (ATCCR HTB-26™) cells were grown in F-12 medium. All cell lines were supplemented with 10% fetal bovine serum (FBS), 1% penicillin streptomycin (PS) and 1% L-Glutamine. In addition, MDA-MB-231 were supplemented with 1% Glucose. For the extended validation panel of BCCLs, the additional cell lines (HCC1428 (ATCC® CRL-2327™), T47D (ATCC® HTB-133™), ZR751 (ATCC® CRL-1500™), ZR-75-30 (ATCC® CRL-1504™), MDA-MB-468 (ATCC® HTB-132™), HCC1143 (ATCC® CRL-2321™), HCC1187 (ATCC® CRL-2322™), BT-549 (ATCC® HTB-122™)) were cultured according to recommended protocols from ATCC. The cell lines were maintained at 37° C. in a humidified atmosphere with 5% CO₂, and all work was performed in a sterile environment. Cells were sub-cultured at approximately 80% confluency by washing with PBS and incubation with trypsin (0.25%) and dividing into new cell culture flasks with fresh medium. Number of cells and viability were calculated using Countess™ Automated Cell Counter (Invitrogen).

### Conditioned Media.

The cell lines were grown to approximately 80% confluency in 175 cm² flasks, washed with PBS three times, and covered with basic medium without additives. The cells were incubated in normal conditions for one hour, before the washing procedure was repeated. Then, 15 mL basic medium was added (no additives) and the cells were incubated for 24 hours at either normoxia (21% O₂, 5% CO₂) or hypoxia (1.2% O₂, 5% CO₂). After 24 hours, the conditioned medium was transferred to tubes and centrifuged at 3000 g for 5 minutes to remove cell debris, and the supernatant was stored at −80° C.

### Enzyme-Linked Immunosorbent Assay.

ELISA was performed on conditioned media for validation of the MS data on vascular endothelial growth factor A (VEGF-A; Quantikine® ELISA Human VEGF Immunoassay, R&D Systems™, DVE00), angiopoietin-like 4 (ANGPTL4; DuoSet® ELISA Development system Human Angiopoietin-like 4, R&D Systems™, DY3485), and cathepsin B (CTSB; DuoSet® ELISA Development system Human Total Cathepsin B, R&D Systems™, DY2176). ELISA analysis was performed after manufacturer's protocol, and results were normalized with total protein concentrations.

### Microdissection of Human Breast Cancer Samples.

Ten micrometer thick formalin-fixed paraffin-embedded (FFPE) sections were deparaffinized, rehydrated and stained with hematoxylin. Breast cancer epithelium and tumor stroma (adjacent non-epithelial tissue) were laser capture micro-dissected (PALM MicroBeam, Zeiss) and pressure catapulted into a tube cap (AdhesiveCap 500 opaque, Zeiss). Tumor epithelium and tumor stroma areas were selected under supervision of an experienced breast pathologist (L.A.A), using digital high-resolution images of parallel sections stained with hematoxylin-eosin. Depending on availability 0.5-1.9×10⁷ μm³ tissue was obtained. Subsequently, to estimate the purity of microdissected samples, we compared the intensities of the epithelial marker cytokeratin-8 in the tumor epithelial and the tumor stroma samples after proteomics analysis. We found on average 62-fold higher intensities of cytokeratin-8 in the tumor epithelium compared to the tumor stroma fraction, respectively (basal-like: 68-fold, p=3.2e-7; luminal-like: 56-fold, p=7.5e-12). By estimation, on average, only 1.6% (median: 1.7%) epithelial tissues was present in the stromal samples. The low levels of epithelium in microdissected stroma was true for both basal-like and luminal-like samples; the luminal-like samples had on average 6.1-fold higher content of cytokeratin-8 compared to basal-like samples in tumor epithelium (p=3.9e-5). This was as expected since cytokeratin-8 is higher in luminal compared with basal-like epithelial cells.

### Sample Preparation and Mass Spectrometry Analysis.

Conditioned media samples were concentrated using 3 kDa Amicon® Ultra-15 Centrifugal Filter Units (Merck, Kenilworth, NJ, USA) and lyophilized using a vacuum concentrator. The protein pellet was dissolved in 8M urea/20 mM methylamine solution and protein concentration was estimated using Qubit™ Protein Assay Kit (Thermo Fisher Scientific). For secretome samples, 10 μg protein from each sample was prepared and total volume was adjusted. Reduction of proteins was performed by adding 4 μL of 100 mM dithiotreitol (DTT), incubating 1 h, room temperature (RT). Followed by alkylation by adding 5 μL of 200 mM iodoacetamide (IAA), incubating for 1 h, RT, in dark. Proteins were digested using a 1:50 ratio of trypsin to protein concentration and incubated overnight at 37° C. The trypsin reaction was stopped by adding 15 μL of 10% formic acid (FA) to each sample. The microdissected patient tissue were prepared with the filter-aided sample preparation (FASP) protocol⁷⁰. In short, the microdissected patient tissue was lysed in 4% SDS, 100 mM DTT and 100 mM Tris/HCl pH8. The lysate was then centrifuged to remove cellular debris and the protein sample was loaded onto a Microcon 30 kDa centrifugal filter unit (Merck Millipore, MA, USA). The samples were washed (8M Urea, 0.1M Tris PH 8.5), alkylated (0.1M IAA) and washed again, first with urea, then three times with 50 mM ammonium bicarbonate. Finally, the proteins were digested on the filter unit using trypsin in a ratio 1:50 trypsin: protein. The resulting peptides were collected by centrifugation. After digestion, all samples were desalted using Oasis HLB mElution plates (Waters, Milford, MA, USA), and lyophilized. Prior to mass spectrometry analysis, conditioned medium samples for discovery experiments were dissolved in 0.1% FA solution, patient samples in 2% acetonitrile (ACN)/0.1% FA solution, and conditioned medium samples for extended validation experiments were dissolved in 5% ACN/5% FA. Peptide concentration of the conditioned media samples was estimated using NanoDrop™.

### LC-Ms/Ms Analysis.

Conditioned media samples from the discovery BCCL panel were analyzed during a 60 min gradient on an LTQ-Orbitrap Elite mass spectrometer (Thermo Fisher Scientific, Waltham, MA, USA) coupled to a Dionex Ultimate 3000 RSLC system. The peptides were separated on a 15 cm×75 μm analytical column (Acclaim PepMap 100 ID nanoViper column) packed with 2 μm C18 beads. The microdissected samples were analyzed in their entirety during a 180 min gradient on a Q-Exative HF mass spectrometry (Thermo Fisher Scientific), coupled to a Dionex Ultimate NCR-3500 RSLC system. The peptides were separated on a 25 cm×75 μm analytical column (PepMap RSLC, EASY-spray column) packed with 2 μm C18 beads). The MS was operated in data-dependent acquisition (DDA) mode. Raw data were acquired through the Xcalibur software (Thermo Fisher Scientific).

Mass spectrometry data for conditioned media samples from the validation BCCL panel were collected using the Exploris 480 mass spectrometer (Thermo Fisher Scientific, San Jose, CA) coupled with a Proxeon 1200 Liquid Chromatograph (Thermo Fisher Scientific). Peptides were separated on a 100 μm inner diameter microcapillary column packed with ˜25 cm of Accucore C18 resin (2.6 μm, 150 Å, Thermo Fisher Scientific). We loaded ˜1 μg onto the column.

Peptides were separated using a 90 min gradient of 3 to 25% acetonitrile in 0.125% formic acid with a flow rate of 520 nL/min. The scan sequence began with an Orbitrap MS¹ spectrum with the following parameters: resolution 120,000, scan range 350-1350 Th, automatic gain control (AGC) target “standard”, maximum injection time “auto”, RF lens setting 40%, and centroid spectrum data type. We selected the top twenty precursors for MS² analysis which consisted of HCD high-energy collision dissociation with the following parameters: resolution 15,000, AGC was set at “standard”, maximum injection time “auto”, isolation window 1.2 Th, normalized collision energy (NCE) 28, and centroid spectrum data type. In addition, unassigned and singly charged species were excluded from MS² analysis and dynamic exclusion was set to 90 s.

### Computational Analysis of Proteomics Data.

Raw MS files from discovery BCCL panel secretomes and micro-dissected tissues were analyzed using MaxQuant⁷¹ (v1.5.3.30 for conditioned medium samples and v1.6.0.16 for patient samples) with label-free quantification and “match between runs” enabled. The precursor ion tolerance for total protein level profiling was set to 20 pmm, and product ion tolerance to 0.5 Da. Carbamidomethylation of cysteines was set as fixed modifications, and oxidation of methionines and N-terminal acetylation was set as variable modifications. False discovery rate (FDR) for peptide and protein identification was set to 1%. MS/MS spectra were searched in the Andromeda search engine against the forward and reverse Human UniProt database.

The validation BCCL panel secretomes, raw data was processed using the FragPipe (v18) proteomics pipeline software, wherein peptide identification was performed with MSFragger (v3.5)⁷² with precursor and fragment mass tolerance in peak matching was set to 20 PPM. Peptide validation was performed with Percolator (v3.05)⁷³, and protein inference was done by ProteinProphet from the Philosopher toolkit (v4.4.0)⁷⁴. MS1 quantification was performed using lonQuant (v1.8)⁷⁵ with the “Match between runs” option enabled. MaxLFQ protein intensity algorithm selected were selected, and intensities were normalized between experiments. Mass-to-charge (m/z) ratio tolerance were set to 10 ppm.

The identified proteins were analyzed using Perseus⁷⁶ (v1.6.0.2 for the discovery BCCL panel secretomes and micro-dissected tissue, and v2.0.7.0 for validation panel BCCL secretomes); the data was grouped into luminal-like or basal-like, and in addition hypoxia or normoxia for conditioned medium samples. Proteins with valid quantification in less than 50% of samples in at least one group were removed for analysis of discovery panel of BCCL and patient samples. Non-filtered data from the extended BCCL panel was used for validation. Imputation was used to replace missing values (from normal distribution: width 0.3, downshift 1.8) for secretome samples in the discovery panel. A two-sample Student's t-test was performed to compare the groups, and a p-value significance threshold was set to 0.05.

Gene ontology analyses were performed using Panther Classification System⁷⁷ (PANTHER14.0, Overrepresentation Test, GO Ontology database released 2019 Jan. 1). Gene sets significantly enriched in the hypoxic secretome were explored by applying the Gene Set Enrichment Analysis (GSEA; www.broadinstitute.org/gsea)⁷⁸ and signatures from Molecular Signatures Database (MSigDB; www.broadinstitute.org/gsea/msigdb) using the fgsea (version 1.15.0) R-package⁷⁹. Protein network analyses were performed using StringDB¹⁵, Cytoscape⁸⁰ (v3.5.1), and the Cytoscape add-on MCODE⁸¹ (v1.4.2). Subcluster analysis was done in MCODE with the following settings: network scoring: include loops: false, degree cutoff: 2; cluster finding: node score cutoff: 0.2, haircut: true, fluff: false, K-Core: 2, max. depth from seed: 100.

The upstream regulator analysis was generated by QIAGEN's Ingenuity Pathway Analysis program (IPA®, QIAGEN Redwood City, www.giagen.com./ingenuity). Settings for IPA were as follows: Expression Analysis with ‘Exp Log Ratio’ values, Reference set (Ingenuity Knowledge Base (Genes Only), Confidence (Experimentally Observed), and for Node Type, Data Source, Species, Tissue & Cell Lines and Mutations, we selected all

### Signature Discovery.

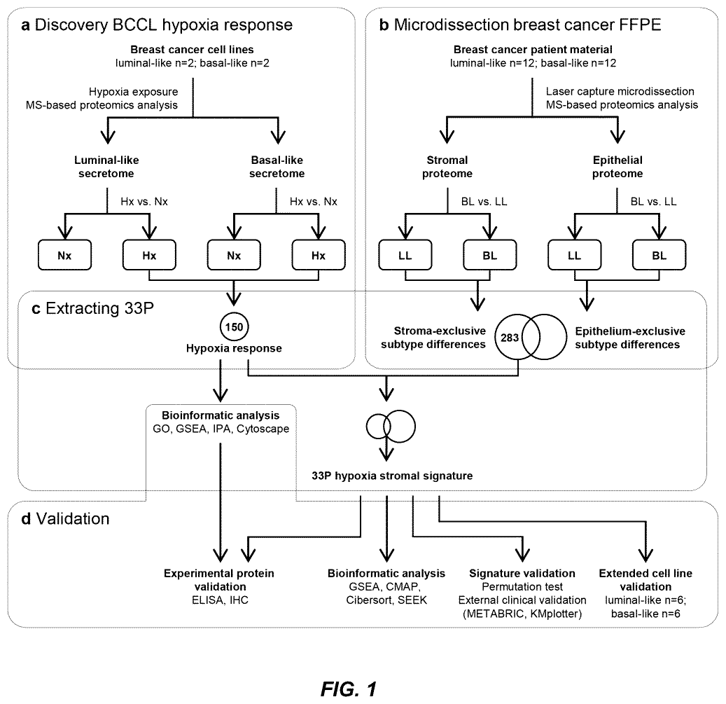

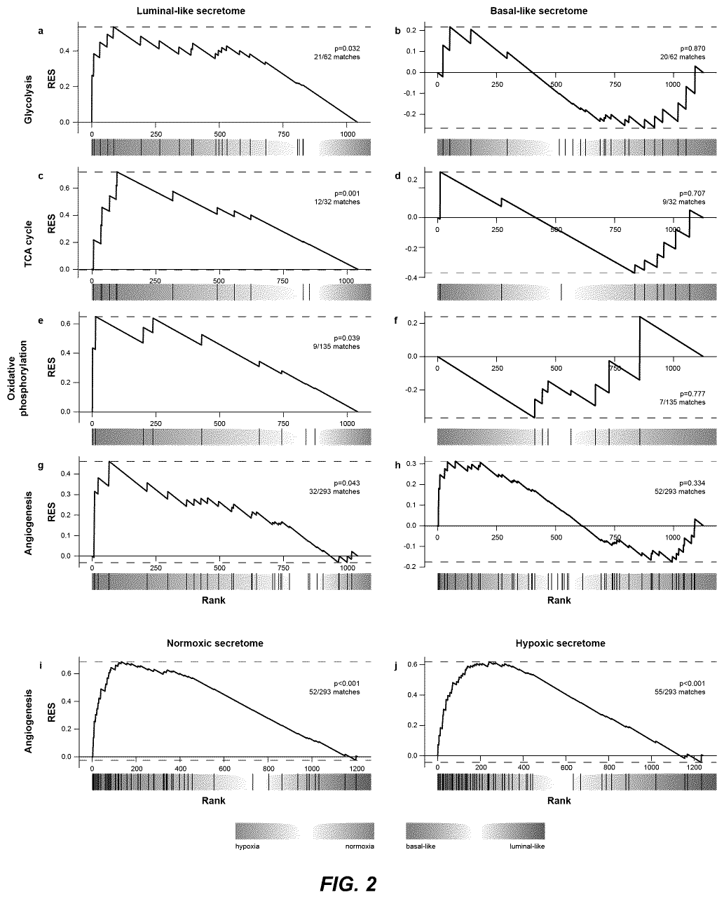

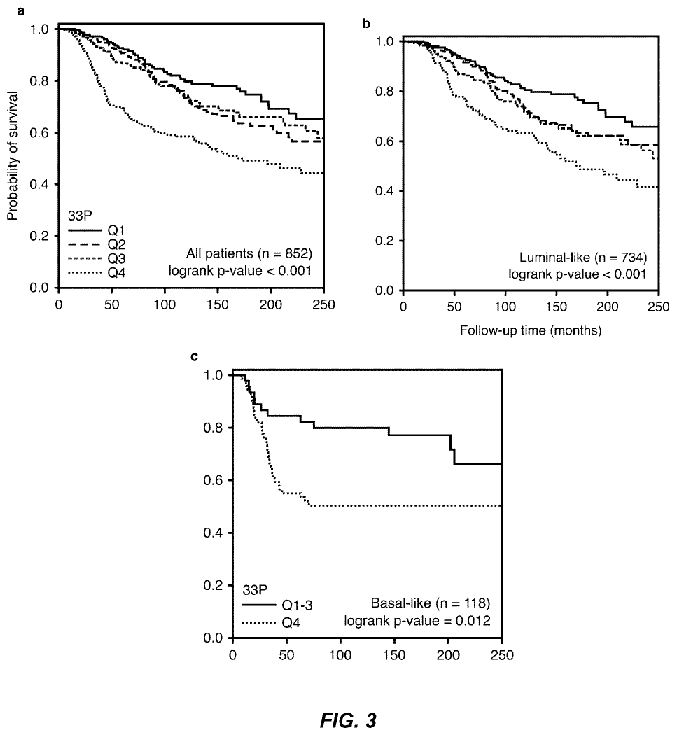

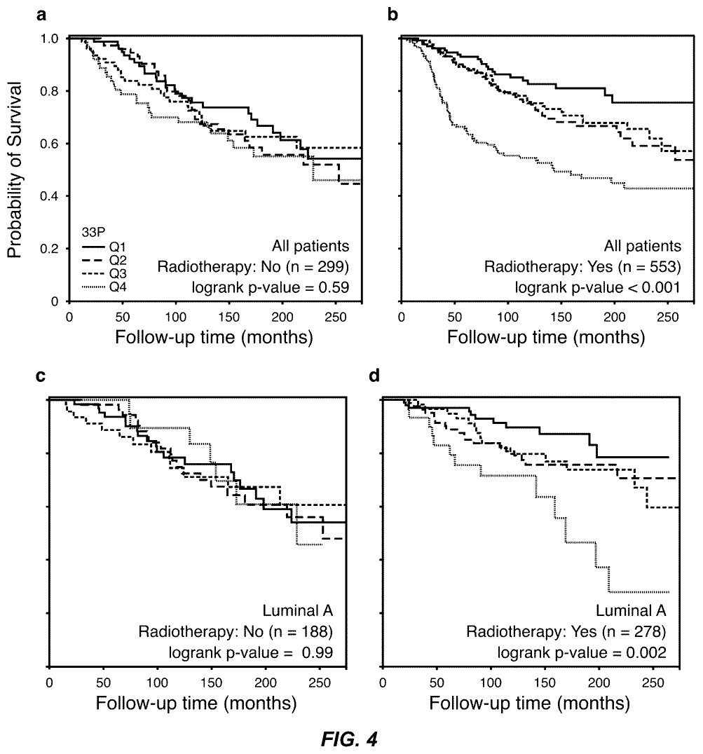

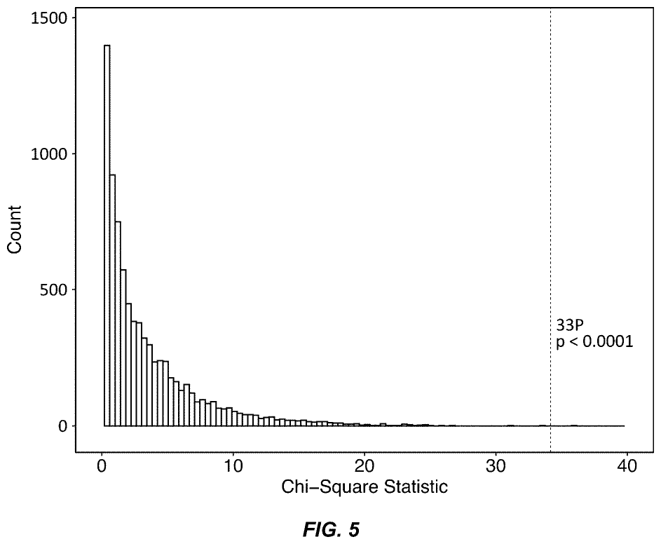

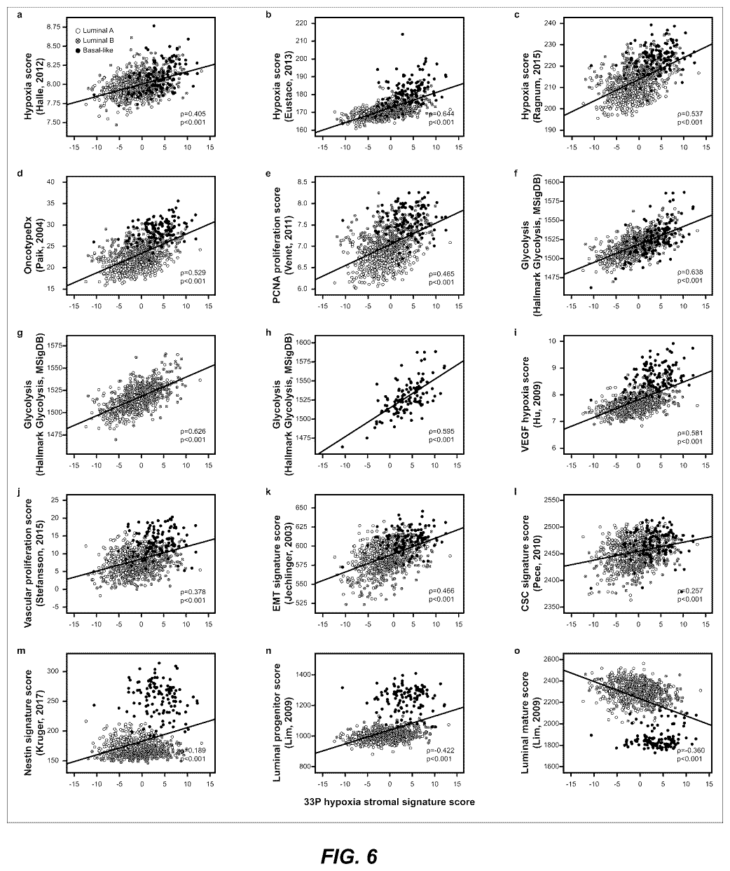

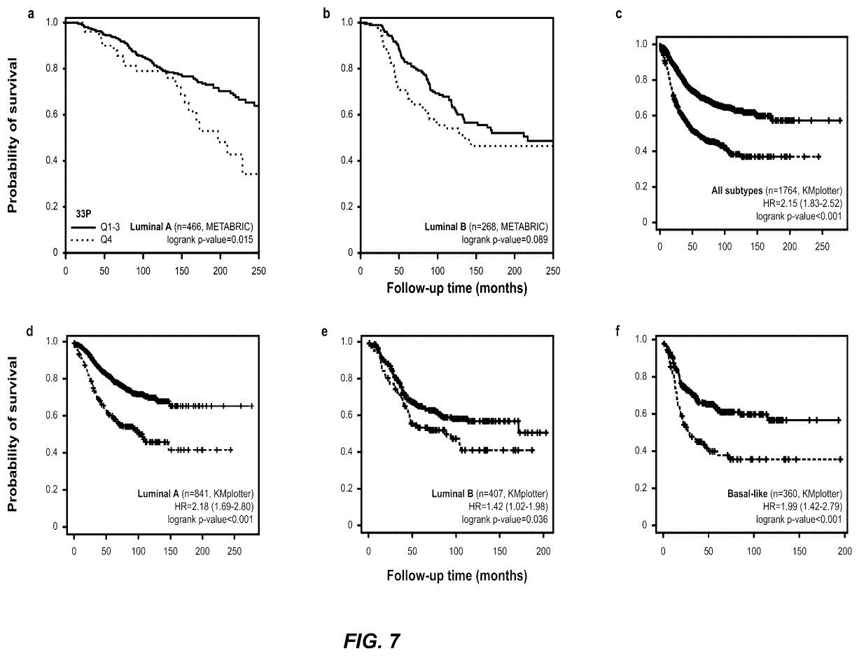

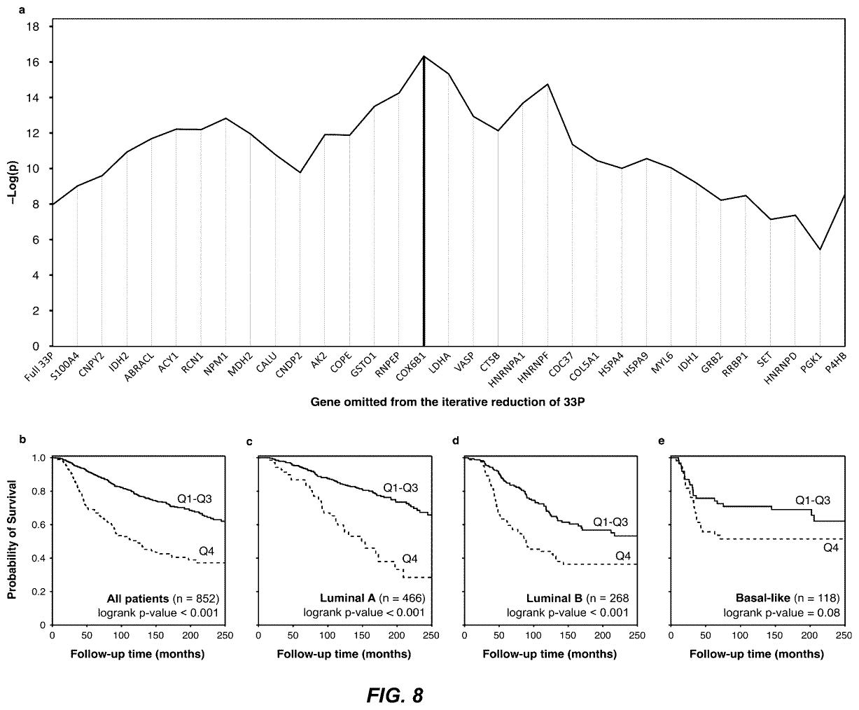

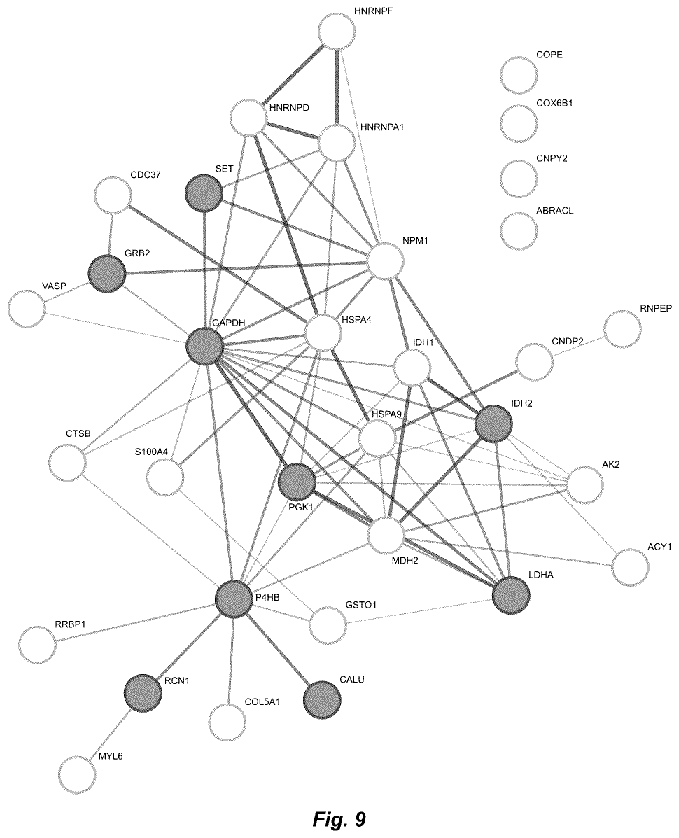

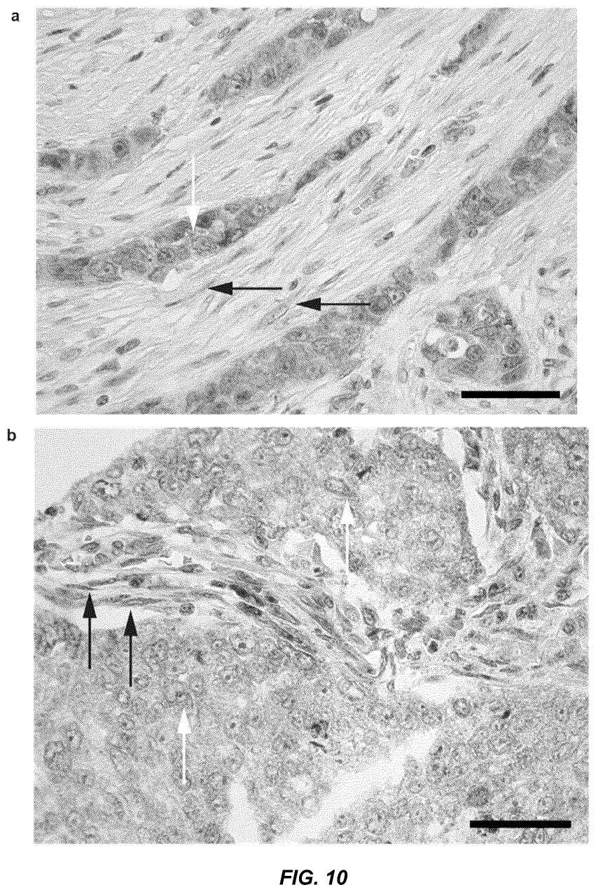

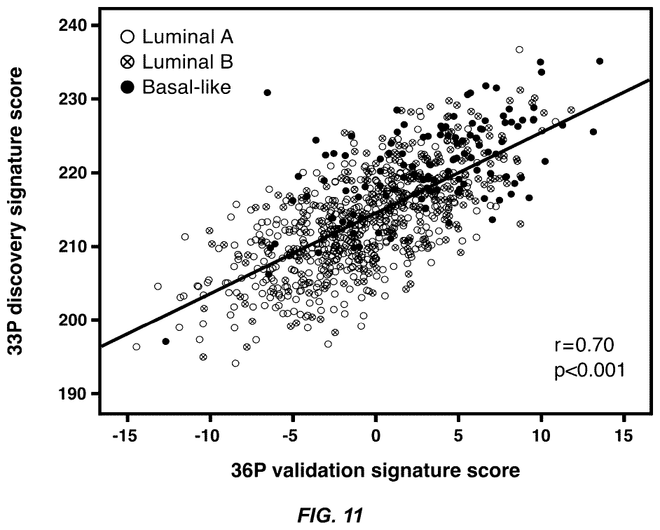

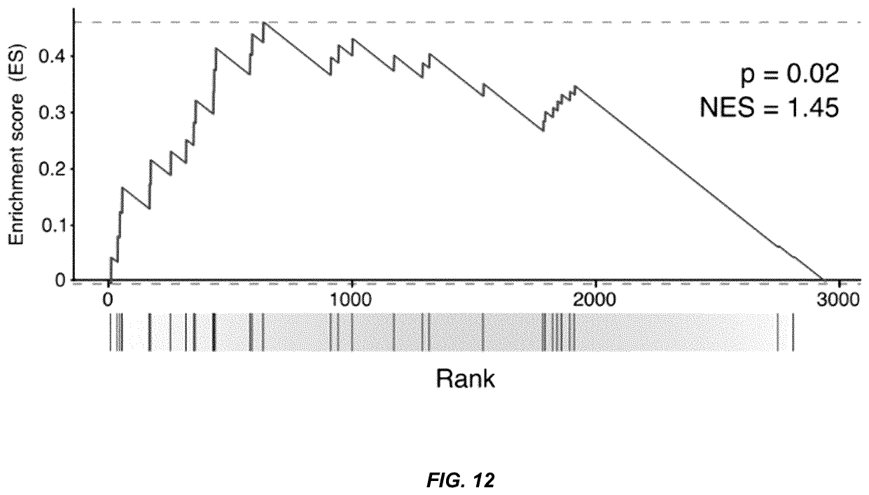

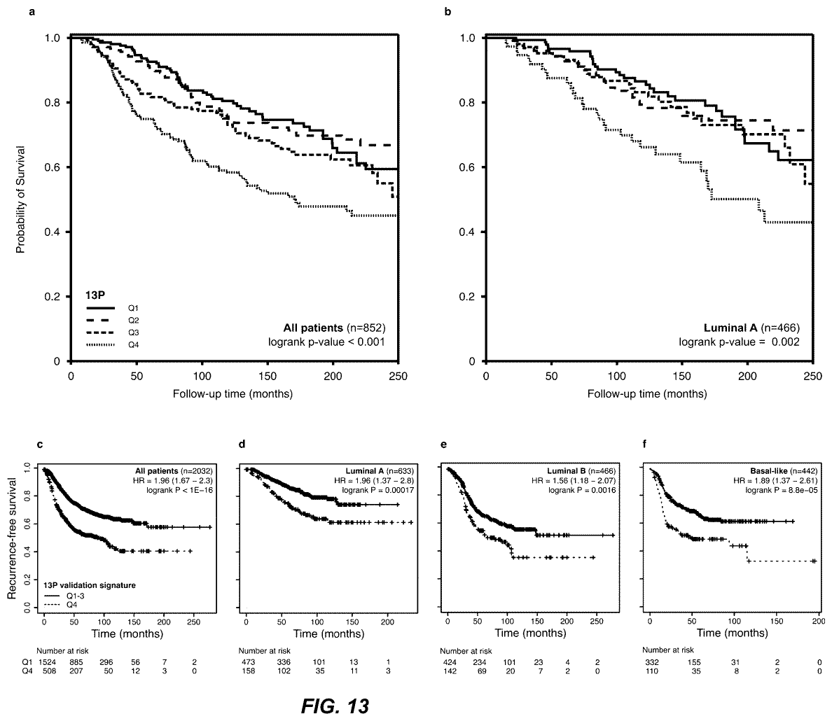

The signature proteins were derived from integrated analysis of secretomes from discovery BCCL and microdissected stromal tissue proteomics data. The proteins that were in common for the hypoxia-increased proteins (hypoxia vs. normoxia) and the stroma-exclusive subtype differences (basal-like vs. luminal-like) were extracted as the protein signature (see FIG. 1c). The signature proteins were validated in the extended validation panel of BCCL.

### Signature Scoring.

Each signature gene was normalized by subtraction, i.e., the average gene expression value (all patient samples) was subtracted from the expression value of each patient sample. The signature score was calculated by summing the normalized expression values for each signature gene.

### Gene Expression Analysis of Patient Cohorts.

For the exploration of gene expression patterns related to the 33P signature score in breast cancer, the signature was mapped to publicly available mRNA datasets with additional information on clinico-pathologic and follow-up data and molecular tumor subtypes, defined by the PAM50 algorithm⁸² (METABRIC-Discovery cohort⁸³, n=852; HER2 and normal-like subtypes were excluded). The online database “KM plotter” (www.kmplot.com)⁸⁴ was also applied to evaluate the 33P mRNA score in relation to recurrence-free breast cancer survival in a merged dataset of 3951 (updated n=4934) breast cancer cases. Cut-off point for analyses (upper quartile) with dichotomized 33P mRNA score values was defined after considering frequency distributions and survival pattern of quartiles.

Gene sets significantly enriched in cases with high 33P mRNA score were explored by applying the Gene Set Enrichment Analysis (GSEA; www.broadinstitute.org/gsea)⁷⁸ and signatures from Molecular Signatures Database (MSigDB; www.broadinstitute.org/gsea/msigdb), using J-Express (version 2012)⁸⁵. Multiple probes covering the same gene were collated according to max probe⁷⁸. Genes differentially expressed between tumors of high versus low 33P mRNA score were identified based on Significance Analysis of Microarrays⁸⁶.

For comparisons, we analyzed separate signature scores reflecting effects of hypoxia²⁰⁻²², scores reflecting proliferation^{23,24}, glycolysis (MSigDB, HALLMARK_GLYCOLYSIS), angiogenesis by vascular proliferation^{25,26}, epithelial-mesenchymal transition (EMT)²⁷, signatures reflecting stemness features^{28,29}, and luminal progenitor and mature luminal signature scores³⁰.

### Connectivity Map Analysis of Drug Signatures.

We explored correlations between the global gene expression pattern of breast cancers with high 33P mRNA score and drug signatures in the Connectivity Map (Cmap) database⁸⁷ (METABRIC-Discovery cohort). As a basis for the Cmap analyses, we included genes differentially expressed (FDR<0.006; fold change ≥1.5 or ≤−1.5) between tumor subsets of low and high 33P mRNA scores (cut-off point upper quartile).

### Proteomic Analysis of External Patients.

We downloaded the recently published proteomic dataset on breast cancer by Asleh and colleagues¹⁹ to explore associations between 33P and clinico-pathologic features (luminal-like and basal-like cancers only; n=209)¹⁹. The 33P signature was scored as described above. The heatmap was generated using the ComplexHeatmap R-package (v2.15.1)⁸⁸. Boxplots were generated using ggplots2.

### Immunohistochemical Staining.

Immunohistochemistry detection of NFR2 expression in tissue samples was performed manually on 4-5 μm thick tissue microarray (TMA) sections from formalin-fixed paraffin-embedded tumor tissue from an in-house cohort of breast cancer patients (n=42; luminal-like 23, basal-like 19) with MS-proteomics information in parallel. Briefly, target retrieval for NRF2 was performed in Ventana Benchmark Ultra staining platform (Roche Tissue Diagnostics, Ventana Medical Systems, USA) with Cell Conditioning (CC1, #06414575001, Roche Tissue Diagnostics, Ventana Medical Systems, USA) (pH9) at 95° C. for 48 minutes before endogenous peroxidases were blocked with Inhibitor CM (from DAB-kit #5266645001, Roche Tissue Diagnostics, Ventana Medical systems) at 37° C. 4 minutes. Slides were incubated with a monoclonal rabbit antibody against NRF2 (Clone EP1808Y, ab62352, Abcam, USA, diluted 1:100) for 60 minutes, followed by incubation with EnVision rabbit HRP (#K400311-2, Agilent, USA) for 30 minutes. To add color at the site of target antigen recognized by the primary antibody, DAB chromogen (#K346811-2, Agilent, USA) was applied for 10 minutes. Finally, sections were rinsed in distilled water and counterstained with Haematoxilin (#S330130-2, Agilent, USA).

NRF2 staining was recorded using a semi-quantitative and subjective grading system, considering the intensity of staining (none=0, weak=1, moderate=2, and strong=3) in tumor stromal and epithelial areas separately⁸⁹. The NRF2 antibody was validated by the manufacturer in both positive and negative cells (HELA) and tissue samples (human pancreatic carcinoma and human kidney cancer tissue) with known localization patterns to confirm specificity and sensitivity, and in-house breast cancer and placenta tissues were established as positive controls.

### Statistical Analyses of Patient Data.

Data were analyzed using SPSS (Statistical Package of Social Sciences), Version 25.0 (Armonk, NY, USA; IBMM, Corp). A two-sided p-value less than 0.05 was considered statistically significant. A p-value of 0.05-0.10 was considered to be of borderline statistical significance (trend). Categories were compared using Pearson's chi-square or Fisher's exact tests when appropriate. Non-parametric correlations of bivariate continuous variables were tested by Spearman's rank correlation test. Spearman's rank correlation coefficient (p) is reported. Mann-Whitney U and Kruskal-Wallis tests were used for comparing continuous variables between groups. Odds ratios (OR) and their 95% confidence intervals were calculated by Mantel-Haenszel method.

For survival analyses, the endpoint was death from breast cancer. Follow-up time was defined as the time from the date of diagnosis to the date of death or last follow-up. Univariate survival analysis by the Kaplan-Meier method was performed using the log-rank test. Patients who died of other causes or who were alive at last date of follow-up were censored. The influence of co-variates on breast cancer specific survival was analyzed by Cox′ proportional hazards multivariate method and tested by the Enter method. All variables were tested by log-minus-log plots to determine their ability to be incorporated in multivariate modelling. When categorizing continuous variables, cut-off points were based on median or quartile values, also considering the distribution profile, the size of subgroups, and number of events in survival analyses.

### Cibersort Analysis.

CIBERSORT¹⁷ is a tool that uses gene expression data to estimate the cell type abundances in a mixed cell population. In our study, we used deconvoluted immune cell type abundances from METABRIC cohort performed by/generated by Craven and colleagues¹⁸.

### Seek Analysis.

Search-Based Exploration of Expression Compendium (SEEK)⁹⁰ is a search engine for transcriptomic data, providing thousands of expression datasets from published studies. SEEK implements a computational method that takes as an input a set of queries, genes, and returns a robust ranking of co-expressed genes whilst it ranks and prioritizes relevant expression datasets. In our study, we used SEEK with the 33P signature as input. We explored the top-ranked datasets, and we used the available information for extra validations of our findings.

### Data Availability.

The mass spectrometry proteomics data generated in this study have been deposited to the ProteomeXchange Consortium⁹¹ via the PRIDE partner repository⁹². The secretome data for the discovery panel of BCCLs are available via ProteomeXchange with the dataset identifier PXD027136. The microdissected patient material data are available via ProteomeXchange with identifier PXD027012. The secretome data for validation panel of BCCLs are available via ProteomeXchange with identifier PXD040532. Mass spectrometry data were searched against the forward and reverse Human reference proteome (UniProtKB) (downloaded 2016 Jan. 8 (discovery BCCL panel), 2022 Nov. 21 (validation BCCL panel), 2017 Oct. 22 (microdissected patient material)). Clinical data on patients used for tissue microdissection might be made available for researchers on a request that does not include revelation of identifiable patient information, upon completion of a Data Transfer Agreement and confirmation of ethical approval. This study included analysis of data from the publicly available METABRIC-Discovery cohort⁸³ (available from the European Genome-Phenome Archive, Dataset ID: EGAD00010000210), and the proteomic dataset from Asleh et al. Nature Communications (2022)¹⁹ available from the supplementary information. Survival analysis for hypoxia signatures was performed using the online KMplotter analysis platform⁸⁴. Publicly available data from the Cancer Cell Line Encyclopedia (CCLE) was used in this study. Processed transcriptomic data from breast cancer cell lines are available from CCLE⁵⁹ and they are accessible via the depmap portal (Broad institute). CCLE proteomic data⁶⁰ are available via the Cancer Cell Line Encyclopedia (CCLE) webpage. CIBERSORT analysis data from Craven et al.¹⁸ are available from the Github user “kelgalla” under the project “tnbctils”. The Search-Based Exploration of Expression Compendium (SEEK) was used for search-based exploration of the identified proteins (publicly available datasets: GSE45255.GPL9693, GSE4922.GPL9694 GSE22093.GPL9695,96, GSE15852.GPL9697). The remaining data are available within the article, supplementary information and source data file.

### Example 1: Hypoxia Response in Breast Cancer

To study the hypoxia response in breast cancer, we first analyzed the secretomes of four selected breast cancer cell lines (BCCL) derived from the two phenotypes (luminal-like, basal-like) by mass spectrometry-based proteomics (FIG. 1a). In the secretomes, we identified a total of 1,787 secreted proteins, across all cell lines and oxygen conditions (see Supplementary Table 1 for details on selected cell lines).

### Example 2: Secretomes are Different Between Breast Cancer Subtypes at Normoxia

We compared the luminal-like and basal-like secretomes at normoxia in the discovery BCCL panel (n=4). The distribution and number of secreted proteins were similar for cells at normoxia and hypoxia (1,211 and 1,245 proteins, respectively). At baseline, 331 proteins showed significantly higher levels from basal-like cell lines compared with luminal-like cells. Conversely, 133 proteins had significantly higher abundance in the luminal-like secretome.

By gene set enrichment analysis (GSEA), processes associated with more aggressive cancer, including metabolic changes, angiogenesis, inflammation, immune responses, tissue remodeling and development, and cellular proliferation, were significantly enriched in the basal-like secretome compared with the luminal-like subtype (all FDR<0.05). This is in line with previously described differences at baseline between luminal-like and basal-like subtypes, based on mRNA and selected individual proteins⁸⁻¹⁰.

Additionally, three of the PAM50 proteins (EGFR, CDH3, SLC39A6) were in common with the proteins separating basal-like and luminal-like secretomes at baseline. Of relevance for secretome studies, we found that 40 of the PAM50 signature genes/proteins have been reported in serum or plasma in the Plasma Proteome Database^{11,12} and/or annotated as “blood protein” in the Human Protein Atlas¹³.

### Example 3: Distinct Hypoxia Responses in Breast Cancer Cells

We focused on secreted proteins being increased by hypoxia (hypoxome); overall 150 proteins were significantly increased as compared to normoxia: 128 in luminal-like and 29 in basal-like cells (Supplementary Data 2). Only 7 proteins overlapped and showed increased abundance in both subtypes after hypoxia: CTSB, GAPDH, HNRNPF, RCN1, RNPEP, SDCBP, and VEGFA. The low number of hypoxia-upregulated proteins in common between the two breast cancer subtypes indicates that luminal-like and basal-like cells have distinct hypoxia responses.

Comparing the proteins separating basal-like and luminal-like secretomes under hypoxia, only one protein was overlapping with the PAM50 gene set (EGFR), indicating that the PAM50 classifier may be lacking hypoxic information for subtype stratification. As we observed several intracellular proteins in our secretomes, we investigated cell viability and found this to be high, with no significant difference between cells conditioned at hypoxia and normoxia (average viability at hypoxia: 92.2%; normoxia: 93.9%), in either the luminal-like (hypoxia: 95.9%; normoxia: 96.5%; p=ns) or basal-like cell lines (hypoxia: 88.5%; normoxia 91.4%; p=ns; Mann-Whitney U test). Further, gene ontology analysis of our secretome proteins showed significant enrichment of proteins in the extracellular region compared to random (GO: 0005576; all proteins, FDR=1.16×10⁻²²⁹).

We then searched for key upstream transcription factors of the combined hypoxia response (luminal-like and basal-like) by the Ingenuity Pathway Analysis (IPA) program. Notably, HIF1A is associated with acute hypoxia response, whereas HIF2A is stabilized in chronic hypoxia¹⁴. Among the top five transcriptional regulators associated with the 150 hypoxia-induced proteins, we found MYC, TP53, ARNT, HNF4A, and HIF1A (ranked by strength of association). We found 15 of the 150 hypoxia-increased proteins to be HIF1A targets.

Next, using the IPA database combined with literature mining, we found that of the 150 hypoxia-increased proteins, Putative phospholipase B-like 2 (PLBD2) have not been previously associated with cancer. Based on sequence similarity, PLBD2 is a putative phospholipase, and probably involved in fatty acid metabolism. Studies are needed to elucidate the role of PLBD2 in cancer. Moreover, 40 of the 150 proteins have not been previously associated with breast cancer.

When IPA was performed separately for luminal-like and basal-like hypoxia responses (upregulated proteins), we found that MYC, TP53, HNF4A and ARNT were the top-ranked upstream transcriptional regulators for the luminal-like response, whereas TP53, ARNT and HIF1A were top-ranked for the basal-like response. Among the top five transcriptional regulators for each subtype, NRF2, encoded by the NFE2L2 gene, was only found in the luminal-like hypoxic secretome, whereas TFEB and BCL6B were exclusively found for the basal-like response. Our findings indicate differences in luminal-like and basal-like hypoxia responses, and that these responses are not exclusively regulated by hypoxia-inducible factors (HIFs).

Next, we investigated a STRING-generated¹⁵ interaction network for the 150 hypoxia-increased proteins, and found higher number of interactions and/or associations compared to a random reference set (PPI enrichment p-value<1.0×10⁻¹⁶) (Supplementary Data 2); 125 of the 150 proteins were associated to at least one other protein in a large main network; the 125 proteins showed overrepresentation of proteins involved in metabolic processes (GOID 8152, p=8.9×10⁻¹⁸) and included angiogenesis (e.g., VEGFA, ANG, ANGPTL4; GOID 1525, p=6.6×10⁻⁴). The network showed subclusters associated with metabolic processes such as glycolysis (e.g., GAPDH, LDHA, MDH2; GOID 6096, p=1.2×10⁻⁴) and TCA cycle (e.g., IDH2, MDH1, ACO1; GOID 6099, p=5.8×10⁻⁹) (Supplementary Table 2).

### Example 4: Hypoxia-Secretomes are Enriched in Proteins Associated with Energy Metabolism

We explored differences in the hypoxia response (upregulated proteins) within luminal-like and basal-like subtypes separately. The luminal-like hypoxome was mainly enriched in processes related to metabolism, such as glycolysis (21 of 62 gene set proteins, p=0.02), TCA cycle (12 of 32 gene set proteins, p=0.04), and oxidative phosphorylation (9 of 35 gene set proteins, p=0.05) (FIG. 2a-f). Lactate dehydrogenase (LDHA), a key enzyme in anaerobic glycolysis, was significantly increased in the hypoxic secretome of luminal-like cells, but not in basal-like cell lines (Supplementary Data 2). Whereas we did not observe significant hypoxia-induced differences in energy metabolism among basal-like cells, these cells still showed 1.9-fold higher levels of LDHA at normoxia compared to the luminal-like hypoxic secretome (p=0.002). In contrast, the basal-like hypoxome showed enrichment related to tissue development, immune responses, inflammation and secretion. Our findings suggest that luminal-like cells have a stronger hypoxia response, while basal-like cells may have adapted to a hypoxic environment in vivo, as hypoxic and necrotic regions are more frequent in rapidly growing tumors, such as basal-like breast cancers.

### Example 5: Hypoxia-Secretomes are Enriched in Proteins Associated with Angiogenesis

Hypoxia is associated with expression and/or secretion of angiogenic proteins being targets of HIFs⁴. We compared angiogenic proteins between hypoxic and normoxic conditions and observed that the global basal-like secretome (normoxic and hypoxic) included 52 angiogenesis-related proteins (GOID 1525), while the luminal-like secretome revealed 32 proteins involved in angiogenesis (FIG. 2g-h). Of the 32 luminal-like matches, 28 were in common with the 52 basal-like angiogenic proteins. The luminal-like, but not the basal-like secretome, showed significant hypoxia-induced enrichment of angiogenesis-related proteins (luminal-like: p=0.043; basal-like: p=0.33). However, several of the basal-like angiogenic proteins were already higher at baseline, compared with hypoxia-increased luminal-like angiogenic proteins, including ANG, NCL, PRCP and VEGFA (Supplementary Data 2).

We then compared angiogenic proteins in luminal-like and basal-like secretomes, both at normoxia and after hypoxia (FIG. 2i-j) and found enrichment of angiogenic proteins in the basal-like secretomes in both oxygen conditions (normoxia: p<0.001; hypoxia: p<0.001). These data indicate discrete angiogenic responses within luminal-like and basal-like cells following hypoxia.

Among the 150 hypoxia-increased proteins, only 8 were associated with angiogenesis (Table 1). Notably, vascular endothelial growth factor A (VEGFA) was the only angiogenesis-related protein that was increased by hypoxia in both luminal-like and basal-like cells. VEGFA showed 3.7-fold higher abundance in normoxic basal-like secretomes compared with hypoxic luminal-like secretomes (p=0.01); this difference was validated by enzyme-linked immunosorbent assay (ELISA) (see also FIG. 1d). Lysosomal Pro-X carboxypeptidase (PRCP), a key regulator of vascular homeostasis, was significantly increased in luminal-like secretomes only, but showed 7.4-fold higher secretion from normoxic basal-like cells compared with hypoxic luminal-like cells (p<0.001). Angiopoietin-like 4 (ANGPTL4) and myosin-9 (MYH9) were the only two angiogenic proteins that were hypoxia-increased only in the basal-like cell lines (Table 1). Secretome levels of ANGPTL4 were evaluated by ELISA for validation and showed the same patterns as in MS data.

Further, cathepsin B (CTSB), being connected to angiogenesis¹⁶, showed higher levels of secretion from baseline basal-like compared to hypoxic luminal-like cell lines, as well as being hypoxia-increased in both subtypes (Supplementary Data 2). This was validated by ELISA and showed similar patterns for different cell lines when examined separately. The luminal-like levels of CTSB were not detected by ELISA, being consistent with the lower secretion from luminal-like than basal-like cells.

Taken together, our data indicate differences in secretion of angiogenic proteins following hypoxia between luminal-like and basal-like cells, with only VEGFA overlapping between subtypes. Luminal-like cells increase their secretion of angiogenesis-promoting factors after hypoxia to a greater extent compared with basal-like cells, although several of the basal-like proteins were considerably higher at baseline. This might suggest that basal-like cancer cells are already in an activated angiogenic-like state at baseline.

### Example 6: Integrated Proteomics Analysis of Cell Line Secretomes and Micro-Dissected Tumor Stroma from Human Breast Cancer

Our data indicate that luminal-like and basal-like cells have distinct hypoxia responses, and that basal-like cells may be characterized by more features related to hypoxia present at baseline. As secreted and released proteins from tumor cells are part of the TME in vivo and important in promoting aggressive TME characteristics, we predicted that such proteins might be identified in the stromal compartment of human breast tumors. We separated tumor stroma and tumor epithelium by laser capture microdissection of formalin-fixed paraffin-embedded luminal-like and basal-like breast cancer samples, followed by shotgun proteomics analysis of the extracted proteins (FIG. 1b). In the tumor cell compartment, 4,157 proteins were detected, compared to 2,150 proteins in stromal samples.

Among stromal proteins, the majority were also found in tumor epithelium. We then focused on proteins that were significantly different between luminal-like and basal-like tumor stroma. Proteins differing significantly between the luminal-like and basal-like subtypes in the tumor epithelium were then subtracted from the set of proteins that differed in the tumor stroma compartment. This resulted in 283 proteins that represented significant and unique differences between the subtypes in the stromal compartment; 202 proteins with significantly higher abundance in basal-like stroma; 81 proteins with significantly higher abundance in luminal-like stroma.

Of interest, six proteins (FOXA1, ERBB2, MAPT, NAT1, PHGDH, KRT5) and one protein (PHGDH) overlapped between PAM50 and the differentially expressed proteins between basal-like and luminal-like subtypes in microdissected tumor epithelium and stroma, respectively. This illustrates that the PAM50 signature is mainly tumor epithelial cell-based.

When exploring the 283 proteins by gene ontology analysis, we found a significant overrepresentation of proteins in the cellular components ‘Extracellular matrix’ (GOID 31012, FDR=6.60×10⁻¹⁵) and ‘Extracellular space’ (GOID 5615, FDR=1.37×10⁻⁵⁷), as well as involvement in processes of ‘Extracellular matrix organization’ (GOID 30198, FDR=5.03×10⁻⁵) and ‘Collagen fibril organization’ (GOID 30199, FDR=7.81×10⁻⁴).

We cross-referenced the 283 proteins with the 150 hypoxia-upregulated proteins from our secretome studies, revealing 33 overlapping proteins that were differentially abundant in both datasets (FIG. 1c). This protein set was termed the 33P hypoxia stromal signature (33P) (Supplementary Table 3). As reflected by the 150 hypoxia-upregulated proteins, 33P was also overrepresented by proteins involved in glycolysis (GOID 6096, FDR=0.0136), TCA cycle (GOID 6099, FDR=0.0132), and other carbohydrate metabolic processes (FDR<0.05).

To examine the uniqueness of this 33P signature compared to a random selection of 33 proteins from a pool of the 150 and 283 proteins (above), we performed a random selection permutation analysis and found that 33P was significantly stronger than expected by random chance (p<0.0001) (FIG. 5) (see also FIG. 1d).

Further, to illuminate potential associations between 33P and specific cell types in the TME, we used Cibersort¹⁷ to deconvolute bulk transcriptomic data from METABRIC-Discovery (n=852). We inferred the immune cell abundance for a subset of patients with basal-like and triple-negative breast cancer¹⁸. Basal-like tumors were stratified using the 33P signature score (Q1-Q3 vs. Q4), and we observed lower number of B-cells and CD8-cells in the worse outcome (Q4) subgroup of 33P, indicating potential immune suppression. Notably, we found fewer resting mast cells and an increase in activated mast cells associated with higher 33P. Our findings indicate an association between 33P and immune cell levels within the basal-like subtype.

As one of the main strengths of secretome studies is the potential presence of such proteins in serum or plasma, we examined the 33P in the PPD^{11,12} and found 32 of the 33 signature proteins (not in PPD: COPE). We further explored the Human Protein Atlas—blood protein¹³ and found all signature proteins to be detected in plasma by MS analysis.

### Example 7: High 33P mRNA Score Associates with Aggressive Breast Cancer Features

We explored whether features of tumor cell hypoxia reflected in the stroma, as indicated by 33P, were associated with aggressive breast cancer phenotypes and patient outcome. For this, we included 852 patients diagnosed with luminal A, luminal B or basal-like breast cancer in the METABRIC-Discovery cohort and extracted normalized expression values (mRNA) of genes corresponding to 33P proteins. High 33P mRNA score (by upper quartile) associated with large tumor size, high histologic grade, lymph node metastases, ER negative tumors, and a basal-like phenotype.

In a recently published proteomics cohort (n=209)¹⁹, the 33P score was significantly associated with molecular breast cancer subtypes. High 33P was associated with high histologic grade (p<0.001; grade 3 vs. 1-2) and high tumor cell proliferation by Ki67 expression (p<0.001).

In addition to basic prognostic factors, 33P correlated with independent signatures and gene sets for tissue hypoxia²⁰⁻²² (FIG. 6); correlations were also significant after removing overlapping proteins (all p<0.001) (Supplementary Table 4) with similar Spearman's rank correlation coefficients (Halle: ρ=0.35, previously ρ=0.40; Eustace: ρ=0.59, previously ρ=0.64). Strong correlations with other hypoxia signatures support that 33P reflects hypoxic features being present in the stromal compartment. A high 33P mRNA score was associated with signatures for proliferation^{23,24}, glycolysis (Hallmark glycolysis, MSigDB), angiogenesis by vascular proliferation^{25,26}, epithelial-to-mesenchymal transition (EMT)²⁷, and stemness²⁸⁻³⁰ (see below, and FIG. 6).

We applied the search-based exploration of expression compendium (SEEK) and found that 33P associated with triple-negative phenotype (ρ=0.0006) and high-grade breast cancer (p<0.00001) in two datasets (GSE45255.GPL96 and GSE4922.GPL96), as well as p53 mutations (GSE22093.GPL96; ρ=0.038); the p53 association was also found in METABRIC-Discovery, including among luminal A cases (ρ=0.02). 33P was higher in tumor tissue compared with normal tissue (GSE15852.GPL96) (ρ=0.001).

### Example 8: High 33P mRNA Score Associates with Reduced Patient Survival

High 33P was associated with decreased breast cancer specific survival (log-rank test, p-value<0.001) (FIG. 3a), also when stratifying by molecular subtype (FIG. 3b-c). Notably, stratification of the luminal-like category showed that high 33P was associated with reduced survival within the luminal A category (log-rank test, p-value=0.02). Conversely, basal-like tumors were significantly stratified by 33P, with clearly better survival for those with lower values (Q1-3). Notably, high 33P was associated with shorter survival in the merged cohorts from KMplotter, and when stratified by subtypes (FIG. 7).

By multivariate survival analysis, 33P demonstrated independent prognostic value when adjusting for molecular subtype (luminal-like or basal-like; by PAM50), as well as the basic prognostic factors tumor diameter, histologic grade and lymph node status (Cox′ regression, Wald test, ρ=0.001) (Table 2), and also when stratifying the cohort by molecular subtype (Supplementary Table 5).

### Example 9: Relation Between 33P and Treatment

To explore the potential interaction between 33P and various treatments, we applied the retrospective observational METABRIC-Discovery cohort (n=852) with information on endocrine treatment, chemotherapy, and radiation therapy. We initially performed stratified survival analyses (with/without treatment), and we found no difference for endocrine treatment or chemotherapy with respect to 33P, while different survival patterns were present for radiation therapy (yes vs. no) (FIG. 4a-b). For those treated with radiotherapy, low 33P (lower quartile) was associated with significantly better survival than high values (upper quartile). Statistically, we found a significant interaction with radiotherapy for the prognostic value of 33P (ρ=0.02; HR=1.93 [1.21-3.30]), also after adjustment for basic factors (tumor diameter, histologic grade, lymph node status). The diverging effect of radiotherapy was significant also in patients with luminal A breast cancer (FIG. 4c-d). Whether 33P might be applied to stratify patients for radiotherapy, would need to be studied in a prospective randomized clinical trial for verification. Of note, patients having radiotherapy had significantly higher histologic grade, and were more often ER negative and lymph node positive.

We then asked whether any of the 33 proteins were more important than others in terms of their impact on patient survival. Using the METABRIC-Discovery dataset (n=852), we applied a reduction algorithm, assuming that not all proteins in 33P would be equally strong. The 33P signature was reduced by recursively leaving one gene/protein out and then testing the predictive strength of the remaining N−1 genes/proteins in a survival analysis (Q1-3 vs. Q4). The strongest N−1 signature (lowest log-rank p-value) was retained, and the process was repeated until only one gene remained. The reduced version of 33P with the strongest effect on survival (p=4.3×10⁻¹⁷, compared to baseline 33P p=1.0×10⁻⁸) was these 18 proteins: CDC37, COL5A1, CTSB, GAPDH, GRB2, HNRNPA1, HNRNPD, HNRNPF, HSPA4, HSPA9, IDH1, LDHA, MYL6, P4HB, PGK1, RRBP1, SET, VASP (FIG. 8). These 18 proteins showed a strong separation of the upper quartile patients (Q4) in the luminal A subgroup, and this prognostic impact was validated in KMplotter (p<1.0×10⁻¹⁶; n=2032), also in the luminal A subgroup (p=0.00015; n=631).

### Example 10: Signatures Reflecting Metabolic Processes, Vascular Proliferation and Cellular Plasticity are Increased in 33P-High Breast Cancer

To investigate and validate the ability of the 33P signature to reflect metabolic reprogramming of the TME, GSEA was performed on the METABRIC-Discovery cohort, with proteins ranked from 33P-high to 33P-low. Gene sets reflecting glycolysis and other metabolic processes were significantly enriched in 33P-high tumors (all FDR<0.05). Glycolysis was overrepresented among 33P proteins (GOID6096, ρ=0.0009), and a gene set reflecting glycolysis was top ranked and significantly enriched in 33P-high tumors by GSEA (rank 2, FDR<0.0002, Hallmark glycolysis, MSigDB) and significantly correlated with 33P in the METABRIC-Discovery cohort (FIG. 6).

A gene set reflecting VEGF signaling was significantly enriched by GSEA in 33P-high tumors (MSigDB, C6 oncogenic signature VEGF_A_UP.V1_DN, FDR<0.0001), and validated by independent signatures reflecting VEGF and vascular proliferation (FIG. 6). Further, 33P significantly correlated with signatures reflecting epithelial-mesenchymal transition and stemness features, including a signature for high Nestin expression²⁷⁻²⁹. Notably, 33P correlated positively with a mammary stem cell score and a luminal progenitor signature, and negatively with a mature luminal signature³⁰ (FIG. 6). Our findings suggest that hypoxia is related to more stem-like features.

We expanded the characterization of 33P by performing a STRING-analysis (string-db.org)¹⁵, and found very strong connectivity between the proteins; 29 of 33 proteins (88%) were included in one large network (FIG. 9). The 150 hypoxia proteins that 33P is derived from also showed high connectivity (83% in one large network). Notably, we found that 9 of the 33P proteins were associated with the “VEGFA-VEGFR2 signaling pathway” (WikiPathways, p<0.001).

Regarding angiogenesis, we have validated our findings using an in-house breast cancer tissue cohort and found that 33P (by MS-proteomics) was positively associated with vascular proliferation by IHC, a marker of activated angiogenesis⁷ (n=42; ρ=0.05).

### Example 11: Gene Expression Propose Compounds with Potential Relevance to 33P-High Breast Cancer

To search for biologically relevant targets in 33P-high breast cancer, we queried the drug signature database Connectivity Map (CMAP version 02)³¹ for compound-related gene expression profiles negatively enriched in 33P-high tumors, as such compounds may contribute to decrease some of the features associated with high 33P scores. Among 1,309 small molecules represented in CMAP, expression profiles from compounds with properties promoting attenuation of tumor effects from hypoxia were top ranked (Supplementary Data 7). Previous studies on many of these compounds have demonstrated anti-hypoxia effects in cancer (e.g., resveratrol³², sirolimus³³). Several of the top-ranked compounds have also been shown to have antioxidant effects and/or effects on the transcription factor NRF2 (nuclear factor erythroid 2-related factor 2), encoded by the NFE2L2 gene (e.g., apigenin³⁴). NRF2, found in our IPA analysis of upstream transcription factors for luminal-like hypoxia response proteins, is a known regulator of genes containing antioxidant response elements^{35,36}

In stratified CMAP analyses (luminal-like and basal-like separately), gene expression profiles of compounds with PI3K/mTOR inhibitory properties were top-ranked and negatively enriched in 33P-high tumors (Supplementary Data 7). Adding to this, signatures reflecting PI3K/AKT/mTOR activation were top-ranked and significantly enriched in tumors with high 33P (mRNA) score (GSEA/MSigDB; H and C6 subsets; FDR<0.05). Taken together, results from CMAP analyses, used as a hypothesis-generating/supporting tool, propose a biological relevance of NRF2 activating and/or PI3K/mTOR inhibitory compounds to 33P-high tumors.

### Example 12: Immunohistochemical Expression of NRF2 in Tumor Tissue

Based on results from IPA and CMAP analyses, IHC was performed to examine NRF2 expression in the tumor stromal and epithelial compartments using a breast cancer cohort of 42 cases with tissue proteomics information and 33P status. Stromal NRF2 expression (FIG. 10) was found to be significantly correlated to the 33P signature scores (ρ=0.56, p<0.001), supporting our IPA findings (above); epithelial NRF2 expression was not associated with 33P.

### Example 13: Validation of 33P by Expanded Cell Line Experiments

To validate 33P derived from the original 4 cell lines (2 luminal-like, 2 basal-like), we added 8 additional cell lines (4 luminal-like, 4 basal-like) in a new validation experiment that included all 12 cell lines (FIG. 1d). We predicted that this expansion would implicate a wider biologic spectrum with increased diversity and better coverage of hypoxia responses. First, we performed a discovery analysis on the validation experiment (12 cell lines), similar to the initial discovery, which resulted in a set of 36 proteins (36P) that correlated significantly with 33P (correlation coefficient 0.70; p<0.001; Pearson) (FIG. 11); the correlation was significant also when overlapping proteins (n=10) were omitted from 36P (correlation coefficient 0.50; p<0.001; Pearson).

Next, we investigated the expression of the 33P proteins in our new dataset (12 cell lines) and found (by GSEA) that 33P was significantly associated with hypoxia (FIG. 12). When examining individual proteins from 33P in the validation dataset, 13 of these proteins were significantly altered in either the luminal-like or basal-like cell lines (Supplementary Table 3). A subscore consisting of these 13 proteins (13P) was generated in the METABRIC-Discovery cohort (mRNA data), showing significant association with tumor subtypes and patient survival among all luminal-like or basal-like breast cancers (n=852), as well as in the luminal A category (n=466) (FIG. 13). This result is similar to what was observed in the initial proteomic discovery data (resulting in 33P), indicating that 13P represents a consistent subset of hypoxia-altered proteins across the entire and expanded cell line panel (n=12). Notably, 13P was slightly stronger than 33P when directly compared in a multivariate analysis of patient survival (METABRIC-Discovery) (Supplementary Data 8), in particular among luminal A cases, indicating that 13P is capturing a broader range of hypoxia responses and might reflect a wider set of aggressive stromal characteristics. The 13P score (Q4, upper quartile) was also associated with reduced survival in the KMplotter dataset (FIG. 13), and 13P was significantly associated with response to radiation therapy (p=0.035, test for interaction). Our data support the validity of the original 33P signature. At the same time, the 13P signature, based on the extended experiments, showed slightly stronger prognostic impact.

SUPPLEMENTARY Table 1
Selected cell lines.

| | | Receptor status | | | Tumor | | |
|---|---|---|---|---|---|---|---|
| Cell line | Subtype | ER | PR | HER2 | type | Source | Literature |

Initial selection (discovery panel)

| | | | | | | | |
|---|---|---|---|---|---|---|---|
| BT-474 | Luminal (B) | + | + | + | IDC | PT | ¹⁻⁵ |
| MCF 7 | Luminal (A) | + | + | − | IDC | PE | ¹⁻⁵ |
| Hs 578T | Basal B | − | − | − | IDC | PT | ¹⁻⁵ |
| | (claudin-low) | | | | | | |
| MDA-MB-231 | Basal B | − | − | − | AC | PE | ¹⁻⁵ |
| | (claudin-low) | | | | | | |

Additionally selected cell lines (validation panel)

| | | | | | | | |
|---|---|---|---|---|---|---|---|
| HCC1428 | Luminal (A) | + | + | − | AC | PE | ^{1-3,5} |
| T47D | Luminal (A) | + | + | − | IDC | PE | ¹⁻⁵ |
| ZR751 | Luminal (A) | + | − | − | IDC | AF | ^{1-3,5} |
| ZR-75-30 | Luminal (B) | + | − | + | IDC | AF | ^{1-3,5} |
| MDA-MB-468 | Basal A | − | − | − | AC | PE | ¹⁻⁵ |
| HCC1143 | Basal A | − | − | − | DC | PT | ^{1-3,5} |
| HCC1187 | Basal A | − | − | − | DC | PT | ^{1-3,5} |
| BT-549 | Basal B | − | − | − | IDC | PT | ^{1,2,5} |
| | (claudin-low) | | | | | | |

AC: adenocarcinoma.
AF: ascites fluid.
DC: ductal carcinoma.
ER: estrogen receptor.
HER2: human epidermal growth factor receptor 2.
IDC: invasive ductal carcinoma.
PE: pleural effusion.
PR: progesterone receptor.
PT: primary tumor.
For additional information, see
Neve et al.¹,
Dai et al.²,
Kao et al.³,
Holliday et al.⁴,
and Nusinow et al.⁵.

TABLE 2
Subclusters of hypoxia-upregulated metabolic
processes.

| Gene | | |
|---|---|---|
| name | Protein name | Subcluster |
| ACO1 ⁽¹⁾ | Cytoplasmic aconitate hydratase | Subcluster 1: |
| FH ⁽¹⁾ | Fumarate hydratase, mitochondrial | 10 nodes, 36 edges |
| FBP1 | Fructose-1,6-bisphosphatase 1 | GOBP*: |
| GOT1 | Aspartate aminotransferase, | Tricarboxylic |
| | cytoplasmic | acid cycle ⁽¹⁾ |
| GPI | Glucose-6-phosphate isomerase | |
| IDH1 ⁽¹⁾ | Isocitrate dehydrogenase | |
| | [NADP] cytoplasmic | |
| IDH2 ⁽¹⁾ | Isocitrate dehydrogenase [NADP], | |
| | mitochondrial | |
| MDH1 ⁽¹⁾ | Malate dehydrogenase, cytoplasmic | |
| ME1 | NADP-dependent malic enzyme | |
| TXNRD1 | Thioredoxin reductase 1, cytoplasmic | |
| CYCS | Cytochrome c | Subcluster 2: |
| GAPDH ⁽¹⁾ | Glyceraldehyde-3-phosphate | 15 nodes, 52 edges |
| | dehydrogenase | GOBP*: Negative |
| HSPA4 | Heat shock 70 kDa protein 4 | regulation of |
| HSPA9 | Stress-70 protein, mitochondrial | apoptosis, |
| HSPH1 | Heat shock protein 105 kDa | glycolysis ⁽¹⁾ |
| LDHA ⁽¹⁾ | L-lactate dehydrogenase A chain | |
| MDH2 ⁽¹⁾ | Malate dehydrogenase, mitochondrial | |
| NME1 | Nucleoside diphosphate kinase A | |
| NME2 | Nucleoside diphosphate kinase B | |
| NPM1 | Nucleophosmin | |
| PRDX4 | Peroxiredoxin-4 | |
| PRDX5 | Peroxiredoxin-5, mitochondrial | |
| SUCLG2 | Succinyl-CoA ligase | |
| | [GDP-forming] subunit | |
| | beta, mitochondrial | |
| TXNDC5 | Thioredoxin domain-containing | |
| | protein 5 | |
| VEGFA | Vascular endothelial growth factor A | |
| AK2 | Adenylate kinase 2, mitochondrial | Subcluster 3: |
| EZR | Ezrin | 7 nodes, 12 edges |
| HSPE1 | 10 kDa heat shock protein, | GOBP*: Membrane |
| | mitochondrial | |
| MSN | Moesin | to membrane |
| P4HB ⁽¹⁾ | Protein disulfide-isomerase | docking, cell redox |
| PGK1 | Phosphoglycerate kinase 1 | homeostasis ⁽¹⁾ |
| TXN ⁽¹⁾ | Thioredoxin | |

GOBP: Gene ontology biological process.
*Biological process significantly overrepresented in subcluster, PANTHER Overrepresentation Test (Released 2018 Nov. 13), GO Ontology database released 2019 Jan. 1. Statistical test: One-sided Fisher's exact test. Adjusting for multiple testing was performed using the Benjamini-Hochberg false discovery rate (FDR) method.
⁽¹⁾ Protein involved in marked enriched biological process for subcluster.

TABLE 3
33P hypoxia stromal signature.

| Protein IDs | Gene name | Stromal subtype | Fold change ⁽¹⁾ |
|---|---|---|---|
| Q03154 | ACY1 | Luminal-like | 2.39 |
| P20908 | COL5A1 ⁽²⁾ | Luminal-like | 1.97 |
| Q9H4A4 | RNPEP ⁽²⁾ | Luminal-like | 1.29 |
| Q9P1F3 | ABRACL | Basal-like | 1.80 |
| P54819 | AK2 ⁽²⁾ | Basal-like | 1.73 |
| O43852 | CALU | Basal-like | 1.69 |
| Q16543 | CDC37 | Basal-like | 1.53 |
| Q96KP4 | CNDP2 | Basal-like | 1.42 |
| Q9Y2B0 | CNPY2 | Basal-like | 2.45 |
| O14579 | COPE | Basal-like | 2.51 |
| P14854 | COX6B1 | Basal-like | 2.23 |
| P07858 | CTSB | Basal-like | 2.89 |
| P04406 | GAPDH ⁽²⁾ | Basal-like | 1.71 |
| P62993 | GRB2 | Basal-like | 2.22 |
| P78417 | GSTO1 ⁽²⁾ | Basal-like | 1.32 |
| P09651 | HNRNPA1 | Basal-like | 1.99 |
| Q14103 | HNRNPD | Basal-like | 1.62 |
| P52597 | HNRNPF ⁽²⁾ | Basal-like | 1.68 |
| P34932 | HSPA4 ⁽²⁾ | Basal-like | 1.83 |
| P38646 | HSPA9 | Basal-like | 1.46 |
| O75874 | IDH1 ⁽²⁾ | Basal-like | 4.86 |
| P48735 | IDH2 | Basal-like | 2.71 |
| P00338 | LDHA ⁽²⁾ | Basal-like | 1.71 |
| P40926 | MDH2 | Basal-like | 2.00 |
| P60660 | MYL6 | Basal-like | 1.37 |
| P06748 | NPM1 ⁽²⁾ | Basal-like | 1.88 |
| P07237 | P4HB | Basal-like | 1.62 |
| P00558 | PGK1 ⁽²⁾ | Basal-like | 1.77 |
| Q15293 | RCN1 | Basal-like | 1.34 |
| Q9P2E9 | RRBP1 | Basal-like | 1.72 |
| P26447 | S100A4 | Basal-like | 1.59 |
| Q01105 | SET ⁽²⁾ | Basal-like | 1.79 |
| P50552 | VASP ⁽²⁾ | Basal-like | 1.89 |

⁽¹⁾ Fold change between luminal-like and basal-like subtype in microdissected
stromal samples.
⁽²⁾ Proteins in 13-protein subsignature of 33P.

TABLE 4
Proteins in common for differentially secreted or
expressed proteins with signatures for breast cancer
subtypes, hypoxia, and stromal features.

| | |
|---|---|
| Overlapping | |
| genes/proteins | Signature/gene set |

Oxygen conditions/hypoxia ⁽¹⁾

| | | |
|---|---|---|
| Breast cancer | GAPDH, AK2 | Halle, 2012 (31 genes) |
| hypoxia | VEGFA, ANGPTL4, | Eustace, 2013 (26 genes) |
| response proteins | LDHA, PGK1 | |
| (150 proteins) | RNASE4 | Ragnum, 2015 (32 genes) |

Stromal hypoxia ⁽²⁾

| | Hypoxia signatures | |
|---|---|---|
| 33P stromal-based | AK2, GAPDH | Halle, 2012 (31 genes) |
| hypoxia signature | LDHA, PGK1 | Eustace, 2013 (26 genes) |
| (33 proteins) | — | Ragnum, 2015 (32 genes) |
| | Proliferation signatures | |
| | GAPDH | OncotypeDx; Paik, 2004 |
| | | (21 genes) |
| | — | PCNA proliferation |
| | | signature; Venet, 2011 |
| | | (131 genes) |
| | Glycolysis | |
| | COL5A1, MDH2, LDHA, | Hallmark glycolysis (200 |
| | IDH1, PGK1 | genes) |
| | Vascular proliferation | |
| | — | Hu, 2009 (13 genes) |
| | — | Stefansson, 2015 (32 |
| | | genes) |
| | EMT and stemness | |
| | — | Jechlinger, 2003 (128 |
| | | genes) |
| | P4HB, GAPDH, AK2 | Pece, 2010 (299 genes) |
| | — | Kruger, 2017 (44 genes) |
| | CTSB | Luminal progenitor |
| | | signature; Lim, 2009 (626 |
| | | genes) |
| | AK2, HNRNPA1 | Mature luminal signature; |
| | | Lim, 2009 (990 genes) |

⁽¹⁾ Oxygen conditions/hypoxia: breast cancer hypoxia response proteins (150 proteins) consist of proteins with increased secretion in response to hypoxia; proteins with significantly higher secretion from hypoxic vs. normoxic breast cancer cell line secretomes. Two-sided Student's t-test, significance level p < 0.05.
⁽²⁾ Stromal hypoxia: 33P stromal-based hypoxia signature (33 proteins) derived from breast cancer hypoxia response proteins and stromal proteome information.

TABLE 5
Multivariate survival analysis (proportional hazards
regression model) stratified by molecular subtype.

| | | Univariate analysis | | Multivariate analysis | |
|---|---|---|---|---|---|
| | | HR | | HR | |
| Variable | n | (95 % CI) | p-value | (95 % CI) | p-value |

Luminal-like subtype (n = 734)
Tumor size

| | | | | | |
|---|---|---|---|---|---|
| ≤20 mm | 228 | 1.00 | | 1.00 | |
| >20 mm | 506 | 2.26 | <0.0005 | 1.89 | <0.0005 |
| | | (1.64-3.11) | | (1.36-2.61) | |

Histologic grade

| | | | | | |
|---|---|---|---|---|---|
| 1-2 | 435 | 1.00 | | 1.00 | |
| 3 | 299 | 1.80 | <0.0005 | 1.46 | 0.005 |
| | | (1.40-2.33) | | (1.12-1.90) | |

Lymph node status

| | | | | | |
|---|---|---|---|---|---|
| Negative | 400 | 1.00 | | 1.00 | |
| Positive | 334 | 2.02 | <0.0005 | 1.68 | <0.0005 |
| | | (1.56-2.63) | | (1.29-2.20) | |

33P hypoxia stromal signature

| | | | | | |
|---|---|---|---|---|---|
| Q123 | 594 | 1.00 | | 1.00 | |
| Q4 | 140 | 1.84 | <0.0005 | 1.57 | 0.003 |
| | | (1.38-2.46) | | (1.17-2.11) | |

Basal-like subtype (n = 118)

| | | | | | |
|---|---|---|---|---|---|
| Tumor size | | | | | |
| ≤20 mm | 35 | 1.00 | | 1.00 | |
| >20 mm | 83 | 0.90 | NS | 0.70 | NS |
| | | (0.49-1.68) | | (0.37-1.31) | |

Histologic grade

| | | | | | |
|---|---|---|---|---|---|
| 1-2 | 8 | 1.00 | | 1.00 | |
| 3 | 110 | 2.04 | NS | 1.47 | NS |
| | | (0.47-8.42) | | (0.35-6.19) | |

Lymph node status

| | | | | | |
|---|---|---|---|---|---|
| Negative | 53 | 1.00 | | 1.00 | |
| Positive | 65 | 2.39 | 0.006 | 2.26 | 0.014 |
| | | (1.28-4.48) | | (1.18-4.33) | |

33P hypoxia stromal signature

| | | | | | |
|---|---|---|---|---|---|
| Q123 | 45 | 1.00 | | 1.00 | |
| Q4 | 73 | 2.28 | 0.014 | 2.10 | 0.030 |
| | | (1.18-4.40) | | (1.08-4.08) | |

CI: confidence interval.
HR: hazard ratio.
n: number of patients.
NS: not significant.
Statistical test: Two-sided Wald test. Adjustment for multiple testing was not performed.

Supplementary Data 2: Proteins significantly upregulated
in response to hypoxia in breast cancer cell lines.

| | | Significant increase | |
|---|---|---|---|
| | | in response | Fold |
| UniProtID | Gene name | to hypoxia in | change |
| Q8NBJ4 | GOLM1 (3)(4) | Luminal-like | 15.35 |
| Q13151 | HNRNPA0 | Luminal-like | 10.48 |
| Q15293 | RCN1 (1)(4) | Luminal-like | 7.16 |
| P49189 | ALDH9A1 | Luminal-like | 6.41 |
| P43490 | NAMPT (4) | Luminal-like | 5.66 |
| P24666 | ACP1 | Luminal-like | 5.58 |
| P19338 | NCL (2)(4) | Luminal-like | 5.46 |
| P15692 | VEGFA (1)(2)(4) | Luminal-like | 5.46 |
| P52788 | SMS | Luminal-like | 5.39 |
| Q9HC84 | MUC5B (3) | Luminal-like | 5.35 |
| P34096 | RNASE4 (4) | Luminal-like | 5.17 |
| P30043 | BLVRB (3) | Luminal-like | 5.06 |
| P30044 | PRDX5 | Luminal-like | 5.03 |
| O43776 | NARS (4) | Luminal-like | 4.79 |
| Q96199 | SUCLG2 | Luminal-like | 4.79 |
| P49327 | FASN | Luminal-like | 4.66 |
| Q9NP97 | DYNLRB1 (3) | Luminal-like | 4.59 |
| P41250 | GARS (4) | Luminal-like | 4.53 |
| O94808 | GFPT2 | Luminal-like | 4.53 |
| P04406 | GAPDH (1)(4) | Luminal-like | 4.47 |
| P13489 | RNH1 | Luminal-like | 4.41 |
| Q92598 | HSPH1 | Luminal-like | 4.38 |
| P21964 | COMT | Luminal-like | 4.38 |
| Q32MZ4 | LRRFIP1 (3) | Luminal-like | 4.32 |
| Q15223 | NECTIN1 (3) | Luminal-like | 4.32 |
| O14579 | COPE | Luminal-like | 4.17 |
| P07919 | UQCRH (4) | Luminal-like | 3.97 |
| O75363 | BCAS1 | Luminal-like | 3.97 |
| Q9NR45 | NANS | Luminal-like | 3.92 |
| P06748 | NPM1 (4) | Luminal-like | 3.89 |
| P54578 | USP14 | Luminal-like | 3.81 |
| O95994 | AGR2 (3) | Luminal-like | 3.73 |
| P48163 | ME1 | Luminal-like | 3.71 |
| P26639 | TARS (4) | Luminal-like | 3.68 |
| Q8N512 | ARRDC1 (3) | Luminal-like | 3.63 |
| P30085 | CMPK1 (4) | Luminal-like | 3.63 |
| Q9H1E3 | NUCKS1 (3) | Luminal-like | 3.61 |
| Q9Y6U3 | SCIN (3) | Luminal-like | 3.53 |
| Q96DG6 | CMBL (3) | Luminal-like | 3.48 |
| Q14103 | HNRNPD | Luminal-like | 3.46 |
| Q92896 | GLG1 (3) | Luminal-like | 3.2 |
| Q99538 | LGMN (4) | Luminal-like | 3.18 |
| O00151 | PDLIM1 | Luminal-like | 2.99 |
| P38646 | HSPA9 (4) | Luminal-like | 2.97 |
| P03950 | ANG (2)(4) | Luminal-like | 2.95 |
| P20810 | CAST (4) | Luminal-like | 2.95 |
| O14773 | TPP1 (4) | Luminal-like | 2.95 |
| P07900 | HSP90AA1 | Luminal-like | 2.93 |
| O75607 | NPM3 | Luminal-like | 2.93 |
| P50897 | PPT1 (4) | Luminal-like | 2.93 |
| P52597 | HNRNPF (1) | Luminal-like | 2.91 |
| Q15691 | MAPRE1 (4) | Luminal-like | 2.89 |
| Q9H993 | ARMT1 (3) | Luminal-like | 2.87 |
| P31937 | HIBADH (4) | Luminal-like | 2.81 |
| P78417 | GSTO1 (4) | Luminal-like | 2.79 |
| Q16543 | CDC37 (4) | Luminal-like | 2.73 |
| Q9P1F3 | ABRACL | Luminal-like | 2.64 |
| Q8IZP2 | ST13P4 | Luminal-like | 2.6 |
| P23381 | WARS (2) | Luminal-like | 2.57 |
| P47813 | EIF1AX | Luminal-like | 2.57 |
| P04066 | FUCA1 | Luminal-like | 2.55 |
| Q01105 | SET | Luminal-like | 2.55 |
| Q00610 | CLTC | Luminal-like | 2.51 |
| P22061 | PCMT1 | Luminal-like | 2.48 |
| P48735 | IDH2 | Luminal-like | 2.43 |
| P63241 | EIF5A (4) | Luminal-like | 2.41 |
| Q9P2E9 | RRBP1 (3) | Luminal-like | 2.41 |
| P48147 | PREP (4) | Luminal-like | 2.41 |
| O43583 | DENR | Luminal-like | 2.38 |
| P23434 | GCSH | Luminal-like | 2.38 |
| P42785 | PRCP (2)(4) | Luminal-like | 2.36 |
| P17096 | HMGA1 (4) | Luminal-like | 2.36 |
| P16930 | FAH | Luminal-like | 2.33 |
| P31949 | S100A11 | Luminal-like | 2.31 |
| Q13630 | TSTA3 | Luminal-like | 2.31 |
| P62316 | SNRPD2 | Luminal-like | 2.31 |
| P07237 | P4HB | Luminal-like | 2.28 |
| P09651 | HNRNPA1 | Luminal-like | 2.27 |
| Q96KP4 | CNDP2 | Luminal-like | 2.25 |
| P14854 | COX6B1 | Luminal-like | 2.19 |
| P54819 | AK2 | Luminal-like | 2.19 |
| Q08257 | CRYZ | Luminal-like | 2.17 |
| P26038 | MSN (4) | Luminal-like | 2.11 |
| Q9H2U2 | PPA2 | Luminal-like | 2.1 |
| Q16881 | TXNRD1 (4) | Luminal-like | 2.06 |
| P35237 | SERPINB6 (3) | Luminal-like | 2.04 |
| P84090 | ERH | Luminal-like | 2.03 |
| O75874 | IDH1 | Luminal-like | 2.03 |
| O00560 | SDCBP (1)(3) | Luminal-like | 2.01 |
| P15311 | EZR | Luminal-like | 1.99 |
| O75223 | GGCT | Luminal-like | 1.99 |
| O43396 | TXNL1 (4) | Luminal-like | 1.97 |
| P10599 | TXN | Luminal-like | 1.96 |
| O75340 | PDCD6 (2) | Luminal-like | 1.96 |
| P99999 | CYCS (4) | Luminal-like | 1.96 |
| P62993 | GRB2 | Luminal-like | 1.95 |
| P07858 | CTSB (1)(4) | Luminal-like | 1.95 |
| P61970 | NUTF2 | Luminal-like | 1.93 |
| O95834 | EML2 (3) | Luminal-like | 1.89 |
| P22392 | NME2 | Luminal-like | 1.89 |
| P00558 | PGK1 | Luminal-like | 1.89 |
| Q9UHV9 | PFDN2 | Luminal-like | 1.88 |
| Q9H4A4 | RNPEP (1) | Luminal-like | 1.88 |
| P56537 | EIF6 (4) | Luminal-like | 1.87 |
| P08195 | SLC3A2 | Luminal-like | 1.84 |
| P09467 | FBP1 | Luminal-like | 1.83 |
| P51858 | HDGF (3) | Luminal-like | 1.8 |
| P40926 | MDH2 | Luminal-like | 1.8 |
| O95394 | PGM3 | Luminal-like | 1.8 |
| P06744 | GPI | Luminal-like | 1.8 |
| P07954 | FH | Luminal-like | 1.77 |
| Q03154 | ACY1 | Luminal-like | 1.77 |
| Q9Y6E2 | BZW2 (3) | Luminal-like | 1.77 |
| P00338 | LDHA (4) | Luminal-like | 1.77 |
| P61604 | HSPE1 | Luminal-like | 1.75 |
| Q9Y2B0 | CNPY2 (3) | Luminal-like | 1.71 |
| Q8NBS9 | TXNDC5 (4) | Luminal-like | 1.67 |
| P15531 | NME1 | Luminal-like | 1.64 |
| Q04760 | GLO1 | Luminal-like | 1.64 |
| P31942 | HNRNPH3 | Luminal-like | 1.56 |
| Q8NHP8 | PLBD2 (3) | Luminal-like | 1.56 |
| P40925 | MDH1 | Luminal-like | 1.56 |
| P34932 | HSPA4 (4) | Luminal-like | 1.54 |
| O00115 | DNASE2 | Luminal-like | 1.52 |
| P17174 | GOT1 | Luminal-like | 1.45 |
| P50395 | GDI2 | Luminal-like | 1.44 |
| P07711 | CTSL (4) | Luminal-like | 1.42 |
| Q96C90 | PPP1R14B (3) | Luminal-like | 1.37 |
| P24593 | IGFBP5 | Basal-like | 48.5 |
| P26447 | S100A4 | Basal-like | 7.94 |
| P01019 | AGT | Basal-like | 6.96 |
| Q9BY76 | ANGPTL4 (2) | Basal-like | 6.15 |
| Q8N114 | SHISA5 (3) | Basal-like | 4.35 |
| P36222 | CHI3L1 | Basal-like | 3.14 |
| P07858 | CTSB (1)(4) | Basal-like | 2.91 |
| P49588 | AARS | Basal-like | 2.83 |
| Q15121 | PEA15 (3) | Basal-like | 2.83 |
| Q14847 | LASP1 | Basal-like | 2.13 |
| O00560 | SDCBP (1)(3) | Basal-like | 2.13 |
| P50552 | VASP | Basal-like | 2.08 |
| P21291 | CSRP1 (3) | Basal-like | 2.07 |
| P20908 | COL5A1 | Basal-like | 2.04 |
| P35579 | MYH9 (2) | Basal-like | 1.99 |
| P15692 | VEGFA (1)(2)(4) | Basal-like | 1.92 |
| P11279 | LAMP1 | Basal-like | 1.83 |
| P52597 | HNRNPF (1) | Basal-like | 1.75 |
| Q96HC4 | PDLIM5 | Basal-like | 1.72 |
| P04406 | GAPDH (1)(4) | Basal-like | 1.71 |
| O43852 | CALU | Basal-like | 1.69 |
| P21399 | ACO1 | Basal-like | 1.67 |
| P60033 | CD81 | Basal-like | 1.58 |
| P60660 | MYL6 | Basal-like | 1.52 |
| Q13162 | PRDX4 | Basal-like | 1.48 |
| P61769 | B2M | Basal-like | 1.42 |
| Q15293 | RCN1 (1)(4) | Basal-like | 1.42 |
| Q9H4A4 | RNPEP (1) | Basal-like | 1.42 |
| P06753 | TPM3 | Basal-like | 1.37 |

1: Proteins upregulated in response to hypoxia in both luminal-like and basal-like breast cancer cell lines.
2: Angiogenesis-related proteins.
3: Single nodes not connected to main network in network biology analysis.
4: Proteins with higher secretion from basal-like vs. luminal-like cell lines at baseline (normoxia).

Supplementary Data 7: Drug signatures negatively correlated to high 33P.

| | | | | | |
|---|---|---|---|---|---|
| | | | | | Mode of |
| | | | | | action/target/ |
| Rank ⁽¹⁾ | CMap name | n | ES | p-value | mechanism ⁽²⁾ |

33P high tumors, all patients

| | | | | | |
|---|---|---|---|---|---|
| 1 | Monobenzone | 4 | −0.96 | 0 | Depigmenting agent. |
| 2 | Resveratrol | 9 | −0.91 | 0 | Antioxidant and anti- |
| | | | | | inflammatory |
| | | | | | compound. |
| 3 | Apigenin | 4 | −0.90 | 0.00022 | Flavonoid. |
| | | | | | Antioxidant and |
| | | | | | anti-inflammatory |
| | | | | | compound. |
| 4 | Trifluoperazine | 16 | −0.50 | 0.00034 | Dopamine antagonist. |
| 5 | Methacholine | 3 | −0.90 | 0.00194 | Non-selective |
| | chloride | | | | muscarinic |
| | | | | | receptor agonist. |
| 6 | Liothyronine | 4 | −0.81 | 0.00251 | Protects against |
| | | | | | oxidative |
| | | | | | stress via PI3K-AKT |
| | | | | | signaling. |
| 7 | Trioxysalen | 4 | −0.81 | 0.00259 | Interstrand DNA |
| | | | | | crosslinking |
| | | | | | after photoactivation. |
| 8 | Acepromazine | 4 | −0.80 | 0.00318 | Dopamine receptor |
| | | | | | antagonist in CNS. |
| 9 | Famprofazone | 6 | −0.67 | 0.00328 | NSAID. Metabolized |
| | | | | | to methamphetamine |
| | | | | | and amphetamine. |
| 10 | Sirolimus | 44 | −0.26 | 0.00435 | mTOR inhibitor. |

33P-high tumors, luminal-like patients

| | | | | | |
|---|---|---|---|---|---|
| 1 | LY-294002 | 61 | −0.42 | 0 | Specific PI3K |
| | | | | | inhibitor, reversible. |
| 2 | Trichostatin A | 182 | −0.24 | 0 | Class I and II |
| | | | | | HDAC inhibitor. |
| 3 | Wortmannin | 18 | −0.48 | 0.00036 | PI3K inhibitor, |
| | | | | | irreversible. |
| 4 | Resveratrol | 9 | −0.64 | 0.0005 | Antioxidant and anti- |
| | | | | | inflammatory |
| | | | | | compound. |
| 5 | Piperidolate | 3 | −0.93 | 0.00062 | Unknown. |
| 6 | Procaine | 5 | −0.75 | 0.00202 | Local anesthetic. |
| | | | | | Benzoic acid derivate. |
| | | | | | Inhibits sodium |
| | | | | | influx. |
| 7 | Monobenzone | 4 | −0.80 | 0.00352 | Depigmenting agent. |
| 8 | Fluphenazine | 18 | −0.41 | 0.00358 | Blocks dopamine D2 |
| | | | | | receptor. |
| 9 | Ethotoin | 6 | −0.66 | 0.00427 | Non-specific |
| | | | | | sodium channel |
| | | | | | blocker. |
| 10 | 0173570-0000 | 6 | −0.66 | 0.00467 | Not tested in humans. |

33P-high tumors, basal-like patients

| | | | | | |
|---|---|---|---|---|---|
| 1 | LY-294002 | 61 | −0.51 | 0 | Specific PI3K |
| | | | | | inhibitor, reversible. |
| 2 | Sirolimus | 44 | −0.40 | 0 | mTOR inhibitor. |
| 3 | Tanespimycin | 62 | −0.27 | 0.00022 | HSP90 inhibitor. |
| 4 | Wortmannin | 18 | −0.47 | 0.00044 | PI3K inhibitor, |
| | | | | | irreversible. |
| 5 | Tyloxapol | 4 | −0.85 | 0.00092 | Mucolytic activity. |
| 6 | DL-thiorphan | 2 | −0.99 | 0.00113 | Inhibitor of |
| | | | | | endopeptidases. |
| 7 | Flufenamic acid | 6 | −0.69 | 0.00232 | NSAID. |
| 8 | Rottlerin | 3 | −0.88 | 0.0031 | Inhibitor of protein |
| | | | | | kinase C. |
| 9 | Hesperetin | 5 | −0.71 | 0.00411 | Antioxidant effect. |
| 10 | Methylergometrine | 4 | −0.77 | 0.00599 | Dopamine D1 |
| | | | | | receptor antagonist. |

n: Number of instances in which the compound was tested in the Connectivity map.
ES: Enrichment score. p-value: The expression changes from the compounds tested were scored according to the 33P high vs. low expression signature and the p-value (two-sided) for each compound represents the distribution of this signature in the n instances as compared with the distribution of this signature among all compounds tested, using a permutation test.
1: Top 10 negatively ranked compounds correlated to 33P-high cases.
2: Mode of action/target/mechanism from PubChem for compound.

Supplementary Data 8
Multivariate survival analysis (proportional hazards regression model)
including 13P hypoxia validation signature in breast cancer patients
(METABRIC-Discovery cohort, n = 852).

| | | Univariate analysis | | Multivariate analysis | |
|---|---|---|---|---|---|
| | | HR | | HR | |
| Variable | n | (95 % CI) | p-value | (95 % CI) | p-value |

All patients (n = 852)
Tumor size

| | | | | | |
|---|---|---|---|---|---|
| <20 mm | 263 | 1.00 | <0.001 | 1.00 | 0.007 |
| >20 mm | 589 | 2.05 | | 1.60 | |
| | | (1.47-2.86) | | (1.13-2.25) | |

Histologic grade

| | | | | | |
|---|---|---|---|---|---|
| 1-2 | 443 | 1.00 | <0.001 | 1.00 | 0.023 |
| 3 | 409 | 1.86 | | 1.40 | |
| | | (1.41-2.46) | | (1.05-1.88) | |

Lymph node status

| | | | | | |
|---|---|---|---|---|---|
| Negative | 453 | 1.00 | <0.001 | 1.00 | <0.001 |
| Positive | 399 | 2.25 | | 1.85 | |
| | | (1.70-2.98) | | (1.39-2.47) | |

33P discovery signature

| | | | | | |
|---|---|---|---|---|---|
| Q1-3 | 639 | 1.00 | <0.001 | 1.00 | 0.077 |
| Q4 | 213 | 2.12 | | 1.39 | |
| | | (1.60-2.81) | | (0.96-2.02) | |

13P validation signature

| | | | | | |
|---|---|---|---|---|---|
| Q1-3 | 639 | 1.00 | <0.001 | 1.00 | 0.041 |
| Q4 | 213 | 2.01 | | 1.46 | |
| | | (1.51-2.67) | | (1.02-2.01) | |

Luminal A (n = 466)
Tumor size

| | | | | | |
|---|---|---|---|---|---|
| <20 mm | 164 | 1.00 | <0.001 | 1.00 | 0.002 |
| >20 mm | 302 | 2.22 | | 1.98 | |
| | | (1.44-3.42) | | (1.28-3.06) | |

Histologic grade

| | | | | | |
|---|---|---|---|---|---|
| 1-2 | 333 | 1.00 | 0.010 | 1.00 | 0.041 |
| 3 | 133 | 1.63 | | 1.48 | |
| | | (1.12-2.37) | | (1.02-2.16) | |

Lymph node status

| | | | | | |
|---|---|---|---|---|---|
| Negative | 273 | 1.00 | 0.014 | 1.00 | 0.092 |
| Positive | 193 | 1.58 | | 1.37 | |
| | | (1.10-2.27) | | (0.95-1.98) | |

33P discovery signature

| | | | | | |
|---|---|---|---|---|---|
| Q1-3 | 414 | 1.00 | 0.017 | 1.00 | 0.848 |
| Q4 | 52 | 1.80 | | 1.06 | |
| | | (1.11-2.92) | | (0.58-1.94) | |

13P validation signature

| | | | | | |
|---|---|---|---|---|---|
| Q1-3 | 391 | 1.00 | <0.001 | 1.00 | 0.041 |
| Q4 | 75 | 2.18 | | 1.99 | |
| | | (1.44-3.31) | | (1.18-3.36) | |

CI: confidence interval.
HR: hazard ratio.
n: number of patients.
NS: not significant.
Statistical test: Two-sided Wald test. Adjustment for multiple testing was not performed.

### REFERENCES

1 Schito, L. & Semenza, G. L. Hypoxia-Inducible Factors: Master Regulators of Cancer Progression. Trends Cancer 2, 758-770 (2016). 2 Wicks, E. E. & Semenza, G. L. Hypoxia-inducible factors: cancer progression and clinical translation. J Clin Invest 132 (2022).
3 Pugh, C. W., Gleadle, J. & Maxwell, P. H. Hypoxia and oxidative stress in breast cancer. Hypoxia signalling pathways. Breast Cancer Res 3, 313-317 (2001).
4 Carmeliet, P. & Jain, R. K. Molecular mechanisms and clinical applications of angiogenesis. Nature 473, 298-307 (2011).
5 Bielenberg, D. R. & Zetter, B. R. The Contribution of Angiogenesis to the Process of Metastasis. Cancer J 21, 267-273 (2015).
6 Semenza, G. L. Molecular mechanisms mediating metastasis of hypoxic breast cancer cells. Trends Mol Med 18, 534-543 (2012).
7 Arnes, J. B. et al. Vascular proliferation is a prognostic factor in breast cancer. Breast Cancer Res Treat 133, 501-510 (2012).
8 Nalwoga, H. et al. Vascular proliferation is increased in basal-like breast cancer. Breast Cancer Research and Treatment 130, 1063-1071 (2011).
9 Sorlie, T. et al. Gene expression patterns of breast carcinomas distinguish tumor subclasses with clinical implications. Proc Natl Acad Sci USA 98, 10869-10874 (2001).
10 Yanovich, G. et al. Clinical Proteomics of Breast Cancer Reveals a Novel Layer of Breast Cancer Classification. Cancer Res 78, 6001-6010 (2018).
11 Muthusamy, B. et al. Plasma Proteome Database as a resource for proteomics research. Proteomics 5, 3531-3536 (2005).
12 Nanjappa, V. et al. Plasma Proteome Database as a resource for proteomics research: 2014 update. Nucleic Acids Res 42, D959-965 (2014).
13 Uhlen, M. et al. The human secretome. Sci Signal 12 (2019).
14 Liu, Q., Palmgren, V. A. C., Danen, E. H. & Le Devedec, S. E. Acute vs. chronic vs. intermittent hypoxia in breast Cancer: a review on its application in in vitro research. Mol Biol Rep 49, 10961-10973 (2022).
15 Szklarczyk, D. et al. The STRING database in 2017: quality-controlled protein-protein association networks, made broadly accessible. Nucleic Acids Res 45, D362-D368 (2017).
16 Aggarwal, N. & Sloane, B. F. Cathepsin B: multiple roles in cancer. Proteomics Clin Appl 8, 427-437 (2014).
17 Newman, A. M. et al. Determining cell type abundance and expression from bulk tissues with digital cytometry. Nat Biotechnol 37, 773-782 (2019).
18 Craven, K. E., Gokmen-Polar, Y. & Badve, S. S. CIBERSORT analysis of TCGA and METABRIC identifies subgroups with better outcomes in triple negative breast cancer. Sci Rep 11, 4691 (2021).
19 Asleh, K. et al. Proteomic analysis of archival breast cancer clinical specimens identifies biological subtypes with distinct survival outcomes. Nat Commun 13, 896 (2022).
20 Halle, C. et al. Hypoxia-induced gene expression in chemoradioresistant cervical cancer revealed by dynamic contrast-enhanced MRI. Cancer Res 72, 5285-5295 (2012).
21 Eustace, A. et al. A 26-gene hypoxia signature predicts benefit from hypoxia-modifying therapy in laryngeal cancer but not bladder cancer. Clin Cancer Res 19, 4879-4888 (2013).
22 Ragnum, H. B. et al. The tumour hypoxia marker pimonidazole reflects a transcriptional programme associated with aggressive prostate cancer. Br J Cancer 112, 382-390 (2015).
23 Paik, S. et al. A multigene assay to predict recurrence of tamoxifen-treated, node-negative breast cancer. N Engl J Med 351, 2817-2826 (2004).
24 Venet, D., Dumont, J. E. & Detours, V. Most random gene expression signatures are significantly associated with breast cancer outcome. PLOS Comput Biol 7, e1002240 (2011).
25 Hu, Z. et al. A compact VEGF signature associated with distant metastases and poor outcomes. BMC Med 7, 9 (2009).
26 Stefansson, I. M. et al. Increased angiogenesis is associated with a 32-gene expression signature and 6p21 amplification in aggressive endometrial cancer. Oncotarget 6, 10634-10645 (2015).
27 Jechlinger, M. et al. Expression profiling of epithelial plasticity in tumor progression. Oncogene 22, 7155-7169 (2003).
28 Pece, S. et al. Biological and molecular heterogeneity of breast cancers correlates with their cancer stem cell content. Cell 140, 62-73 (2010).
29 Kruger, K. et al. Expression of Nestin associates with BRCA1 mutations, a basal-like phenotype and aggressive breast cancer. Sci Rep 7, 1089 (2017).
30 Lim, E. et al. Aberrant luminal progenitors as the candidate target population for basal tumor development in BRCA1 mutation carriers. Nat Med 15, 907-913 (2009).
31 Subramanian, A. et al. A Next Generation Connectivity Map: L1000 Platform and the First 1,000,000 Profiles. Cell 171, 1437-1452 e1417 (2017).
32 Zhang, Q. et al. Resveratrol inhibits hypoxia-induced accumulation of hypoxia-inducible factor-1alpha and VEGF expression in human tongue squamous cell carcinoma and hepatoma cells. Mol Cancer Ther 4, 1465-1474 (2005).
33 Perl, A. mTOR activation is a biomarker and a central pathway to autoimmune disorders, cancer, obesity, and aging. Ann N Y Acad Sci 1346, 33-44 (2015).
34 Salehi, B. et al. The Therapeutic Potential of Apigenin. Int J Mol Sci 20 (2019).
35 Rojo de la Vega, M., Chapman, E. & Zhang, D. D. NRF2 and the Hallmarks of Cancer. Cancer Cell 34, 21-43 (2018).
36 Sajadimajd, S. & Khazaei, M. Oxidative Stress and Cancer: The Role of Nrf2. Curr Cancer Drug Targets 18, 538-557 (2018).
37 Vinaiphat, A., Low, J. K., Yeoh, K. W., Chng, W. J. & Sze, S. K. Application of Advanced Mass Spectrometry-Based Proteomics to Study Hypoxia Driven Cancer Progression. Front Oncol 11, 559822 (2021).
38 Cox, T. R. et al. The hypoxic cancer secretome induces pre-metastatic bone lesions through lysyl oxidase. Nature 522, 106-110 (2015).
39 Yoon, J. H. et al. Proteomic analysis of hypoxia-induced U373MG glioma secretome reveals novel hypoxia-dependent migration factors. Proteomics 14, 1494-1502 (2014).
40 Maia, J., Caja, S., Strano Moraes, M. C., Couto, N. & Costa-Silva, B. Exosome-Based Cell-Cell Communication in the Tumor Microenvironment. Front Cell Dev Biol 6, 18 (2018).
41 Rankin, E. B. & Giaccia, A. J. Hypoxic control of metastasis. Science 352, 175-180 (2016).
42 Raimundo, N., Baysal, B. E. & Shadel, G. S. Revisiting the TCA cycle: signaling to tumor formation. Trends in Molecular Medicine 17, 641-649 (2011).
43 Liberti, M. V. & Locasale, J. W. The Warburg Effect: How Does it Benefit Cancer Cells? Trends Biochem Sci 41, 211-218 (2016).
44 Warburg, O., Wind, F. & Negelein, E. The Metabolism of Tumors in the Body. J Gen Physiol 8, 519-530 (1927).
45 Chaffer, C. L. et al. Poised chromatin at the ZEB1 promoter enables breast cancer cell plasticity and enhances tumorigenicity. Cell 154, 61-74 (2013).
46 Miller, L. D. et al. Immunogenic Subtypes of Breast Cancer Delineated by Gene Classifiers of Immune Responsiveness. Cancer Immunol Res 4, 600-610 (2016).
47 Toft, D. J. & Cryns, V. L. Minireview: Basal-like breast cancer: from molecular profiles to targeted therapies. Molecular endocrinology 25, 199-211 (2011).
48 Begg, K. & Tavassoli, M. Inside the hypoxic tumour: reprogramming of the DDR and radioresistance. Cell Death Discov 6, 77 (2020).
49 Godet, I. et al. Fate-mapping post-hypoxic tumor cells reveals a ROS-resistant phenotype that promotes metastasis. Nat Commun 10, 4862 (2019).
50 Shweiki, D., Itin, A., Soffer, D. & Keshet, E. Vascular endothelial growth factor induced by hypoxia may mediate hypoxia-initiated angiogenesis. Nature 359, 843-845 (1992).
51 Hanahan, D. & Folkman, J. Patterns and emerging mechanisms of the angiogenic switch during tumorigenesis. Cell 86, 353-364 (1996).
52 Nalwoga, H. et al. Vascular proliferation is increased in basal-like breast cancer. Breast Cancer Res Treat 130, 1063-1071 (2011).
53 Kruger, K. et al. Microvessel proliferation by co-expression of endothelial nestin and Ki-67 is associated with a basal-like phenotype and aggressive features in breast cancer. Breast 22, 282-288 (2013).
54 Kuo, I. Y., Hsieh, C. H., Kuo, W. T., Chang, C. P. & Wang, Y. C. Recent advances in conventional and unconventional vesicular secretion pathways in the tumor microenvironment. J Biomed Sci 29, 56 (2022).
55 Horsman, M. R., Mortensen, L. S., Petersen, J. B., Busk, M. & Overgaard, J. Imaging hypoxia to improve radiotherapy outcome. Nat Rev Clin Oncol 9, 674-687 (2012).
56 Graham, K. & Unger, E. Overcoming tumor hypoxia as a barrier to radiotherapy, chemotherapy and immunotherapy in cancer treatment. Int J Nanomedicine 13, 6049-6058 (2018).
57 Fruman, D. A. et al. The PI3K Pathway in Human Disease. Cell 170, 605-635 (2017).
58 Gonzalez-Angulo, A. M. et al. Open-label randomized clinical trial of standard neoadjuvant chemotherapy with paclitaxel followed by FEC versus the combination of paclitaxel and everolimus followed by FEC in women with triple receptor-negative breast cancerdagger. Ann Oncol 25, 1122-1127 (2014).
59 Ghandi, M. et al. Next-generation characterization of the Cancer Cell Line Encyclopedia. Nature 569, 503-508 (2019).
60 Nusinow, D. P. et al. Quantitative Proteomics of the Cancer Cell Line Encyclopedia. Cell 180, 387-402 e316 (2020).
61 Neve, R. M. et al. A collection of breast cancer cell lines for the study of functionally distinct cancer subtypes. Cancer Cell 10, 515-527 (2006).
62 Dai, X., Cheng, H., Bai, Z. & Li, J. Breast Cancer Cell Line Classification and Its Relevance with Breast Tumor Subtyping. J Cancer 8, 3131-3141 (2017).
63 Kao, J. et al. Molecular profiling of breast cancer cell lines defines relevant tumor models and provides a resource for cancer gene discovery. PLOS One 4, e6146 (2009).
64 Holliday, D. L. & Speirs, V. Choosing the right cell line for breast cancer research. Breast Cancer Res 13, 215 (2011).
65 Lehmann, B. D. et al. Identification of human triple-negative breast cancer subtypes and preclinical models for selection of targeted therapies. J Clin Invest 121, 2750-2767 (2011).
66 Prat, A. et al. Characterization of cell lines derived from breast cancers and normal mammary tissues for the study of the intrinsic molecular subtypes. Breast Cancer Res Treat 142, 237-255 (2013).
67 Perou, C. M. et al. Molecular portraits of human breast tumours. Nature 406, 747-752 (2000).
68 Goldhirsch, A. et al. Personalizing the treatment of women with early breast cancer: highlights of 68 the St Gallen International Expert Consensus on the Primary Therapy of Early Breast Cancer 2013. Ann Oncol 24, 2206-2223 (2013).
69 Knutsvik, G. et al. Evaluation of Ki67 expression across distinct categories of breast cancer specimens: a population-based study of matched surgical specimens, core needle biopsies and tissue microarrays. PLOS One 9, e112121 (2014).
70 Wisniewski, J. R. Proteomic sample preparation from formalin fixed and paraffin embedded tissue. J Vis Exp (2013).
71 Cox, J. & Mann, M. MaxQuant enables high peptide identification rates, individualized p.p.b.-range mass accuracies and proteome-wide protein quantification. Nat Biotechnol 26, 1367-1372 (2008).
72 Kong, A. T., Leprevost, F. V., Avtonomov, D. M., Mellacheruvu, D. & Nesvizhskii, A. I. MSFragger: ultrafast and comprehensive peptide identification in mass spectrometry-based proteomics. Nature methods 14, 513-520 (2017).
73 Kall, L., Canterbury, J. D., Weston, J., Noble, W. S. & MacCoss, M. J. Semi-supervised learning for peptide identification from shotgun proteomics datasets. Nature methods 4, 923-925 (2007).
74 da Veiga Leprevost, F. et al. Philosopher: a versatile toolkit for shotgun proteomics data analysis. Nature methods 17, 869-870 (2020).
75 Yu, F., Haynes, S. E. & Nesvizhskii, A. I. IonQuant Enables Accurate and Sensitive Label-Free Quantification With FDR-Controlled Match-Between-Runs. Molecular & cellular proteomics: MCP 20, 100077 (2021).
76 Tyanova, S. et al. The Perseus computational platform for comprehensive analysis of (prote) omics data. Nat Methods 13, 731-740 (2016).
77 Mi, H., Muruganujan, A., Ebert, D., Huang, X. & Thomas, P. D. PANTHER version 14: more genomes, a new PANTHER GO-slim and improvements in enrichment analysis tools. Nucleic Acids Res 47, D419-D426 (2019).
78 Subramanian, A. et al. Gene set enrichment analysis: a knowledge-based approach for interpreting genome-wide expression profiles. Proc Natl Acad Sci USA 102, 15545-15550 (2005).
79 Korotkevich, G. et al. Fast gene set enrichment analysis. bioRxiv, 060012 (2021).
80 Shannon, P. et al. Cytoscape: a software environment for integrated models of biomolecular interaction networks. Genome Res 13, 2498-2504 (2003).
81 Bader, G. D. & Hogue, C. W. An automated method for finding molecular complexes in large protein interaction networks. BMC Bioinformatics 4, 2 (2003).
82 Parker, J. S. et al. Supervised risk predictor of breast cancer based on intrinsic subtypes. J Clin Oncol 27, 1160-1167 (2009).
83 Curtis, C. et al. The genomic and transcriptomic architecture of 2,000 breast tumours reveals novel subgroups. Nature 486, 346-352 (2012).
84 Gyorffy, B. et al. An online survival analysis tool to rapidly assess the effect of 22,277 genes on breast cancer prognosis using microarray data of 1,809 patients. Breast Cancer Res Treat 123, 725-731 (2010).
85 Dysvik, B. & Jonassen, I. J-Express: exploring gene expression data using Java. Bioinformatics 17, 369-370 (2001).
86 Tusher, V. G., Tibshirani, R. & Chu, G. Significance analysis of microarrays applied to the ionizing radiation response. Proc Natl Acad Sci USA 98, 5116-5121 (2001).
87 Lamb, J. et al. The Connectivity Map: using gene-expression signatures to connect small molecules, genes, and disease. Science 313, 1929-1935 (2006).
88 Gu, Z., Eils, R. & Schlesner, M. Complex heatmaps reveal patterns and correlations in multidimensional genomic data. Bioinformatics 32, 2847-2849 (2016).
89 Askeland, C. et al. Stathmin expression associates with vascular and immune responses in aggressive breast cancer subgroups. Sci Rep 10, 2914 (2020).
90 Zhu, Q. et al. Targeted exploration and analysis of large cross-platform human transcriptomic compendia. Nat Methods 12, 211-214, 213 p following 214 (2015).
91 Vizcaino, J. A. et al. ProteomeXchange provides globally coordinated proteomics data submission and dissemination. Nature biotechnology 32, 223-226 (2014).
92 Vizcaino, J. A. et al. The PRoteomics IDEntifications (PRIDE) database and associated tools: status in 2013. Nucleic acids research 41, D1063-1069 (2013).
93 Nagalla, S. et al. Interactions between immunity, proliferation and molecular subtype in breast cancer prognosis. Genome Biol 14, R34 (2013).
94 Ivshina, A. V. et al. Genetic reclassification of histologic grade delineates new clinical subtypes of breast cancer. Cancer Res 66, 10292-10301 (2006).
95 Iwamoto, T. et al. Gene pathways associated with prognosis and chemotherapy sensitivity in molecular subtypes of breast cancer. J Natl Cancer Inst 103, 264-272 (2011).
96 Shen, K. et al. A systematic evaluation of multi-gene predictors for the pathological response of breast cancer patients to chemotherapy. PLOS One 7, e49529 (2012).
97 Pau Ni, I. B. et al. Gene expression patterns distinguish breast carcinomas from normal breast tissues: the Malaysian context. Pathol Res Pract 206, 223-228 (2010).

### Supplementary References (from Supplementary Figures and Tables)

S1 Neve, R. M. et al. A collection of breast cancer cell lines for the study of functionally distinct cancer subtypes. Cancer Cell 10, 515-527 (2006).
S2 Dai, X., Cheng, H., Bai, Z. & Li, J. Breast Cancer Cell Line Classification and Its Relevance with Breast Tumor Subtyping. J Cancer 8, 3131-3141 (2017).
S3 Kao, J. et al. Molecular profiling of breast cancer cell lines defines relevant tumor models and provides a resource for cancer gene discovery. PLOS One 4, e6146 (2009).
S4 Holliday, D. L. & Speirs, V. Choosing the right cell line for breast cancer research. Breast Cancer Res 13, 215 (2011).
S5 Nusinow, D. P. et al. Quantitative Proteomics of the Cancer Cell Line Encyclopedia. Cell 180, 387-402 e316 (2020).

1. A method of treating breast cancer in a subject, the method comprising the steps of:
(a) administering a treatment appropriate for treating breast cancer to the subject, wherein, prior to administration, a signature score which was produced from normalised levels of at least 3 biomarkers, wherein the at least 3 biomarkers were selected from a first group consisting of GAPDH, HSPA4, LDHA and VASP, and wherein the at least 3 biomarkers were obtained from a biological sample which was obtained from the subject, had been determined to be above a reference signature score.

2. The method as claimed in claim 1, wherein the signature score was produced from normalised levels of at least 5 biomarkers, wherein the at least 5 biomarkers were selected from a second group consisting of COL5A1, GAPDH, NRNPF, HSPA4, IDH1, LDHA, PGK1, SET and VASP.

3. The method as claimed in claim 1, wherein the signature score was produced from normalised levels of at least 7 biomarkers, wherein the at least 7 biomarkers were selected from a second group consisting of COL5A1, GAPDH, HNRNPF, HSPA4, IDH1, LDHA, PGK1, SET and VASP.

4. The method as claimed in claim 1, wherein the signature score was produced from normalised levels of all of the 9 biomarkers COL5A1, GAPDH, HNRNPF, HSPA4, IDH1, LDHA, PGK1, SET and VASP.

5. The method as claimed in claim 1, wherein the signature score was produced from normalised levels of at least 5 biomarkers, wherein the at least 5 biomarkers were selected from a third group consisting of COL5A1, RNPEP, AK2, GAPDH, GSTO1, HNRNPF, HSPA4, IDH1, LDHA, NPM1, PGK1, SET and VASP.

6. The method as claimed in claim 1, wherein the signature score was produced from normalised levels of at least 10 biomarkers, wherein the at least 10 biomarkers were selected from a third group consisting of COL5A1, RNPEP, AK2, GAPDH, GSTO1, HNRNPF, HSPA4, IDH1, LDHA, NPM1, PGK1, SET and VASP.

7. The method as claimed in claim 1, wherein the signature score was produced from normalised levels of all of the 13 biomarkers COL5A1, RNPEP, AK2, GAPDH, GSTO1, HNRNPF, HSPA4, IDH1, LDHA, NPM1, PGK1, SET and VASP.

8. The method as claimed in claim 1, wherein the signature score was produced from normalised levels of at least 5 biomarkers, wherein the at least 5 biomarkers were selected from a fourth group consisting of CDC37, COL5A1, CTSB, GAPDH, GRB2, HNRNPA1, HNRNPD, HNRNPF, HSPA4, HSPA9, IDH1, LDHA, MYL6, P4HB, PGK1, RRBP1, SET and VASP.

9. The method as claimed in claim 1, wherein the signature score was produced from normalised levels of at least 15 biomarkers, wherein the at least 15 biomarkers were selected from a fourth group consisting of CDC37, COL5A1, CTSB, GAPDH, GRB2, HNRNPA1, HNRNPD, HNRNPF, HSPA4, HSPA9, IDH1, LDHA, MYL6, P4HB, PGK1, RRBP1, SET and VASP.

10. The method as claimed in claim 1, wherein the signature score was produced from normalised levels of all of the 18 biomarkers CDC37, COL5A1, CTSB, GAPDH, GRB2, HNRNPA1, HNRNPD, HNRNPF, HSPA4, HSPA9, IDH1, LDHA, MYL6, P4HB, PGK1, RRBP1, SET and VASP.

11. The method as claimed in claim 1, wherein the signature score was produced from normalised levels of at least 15 biomarkers, wherein the at least 15 biomarkers were selected from a fifth group consisting of ACY1, COL5A1, RNPEP, ABRACL, AK2, CALU, CDC37, CNDP2, CNPY2, COPE, COX6B1, CTSB, GAPDH, GRB2, GSTO1, HNRNPA1, HNRNPD, HNRNPF, HSPA4, HSPA9, IDH1, IDH2, LDHA, MDH2, MYL6, NPM1, P4HB, PGK1, RCN1, RRBP1, S100A4, SET and VASP.

12. The method as claimed in claim 1, wherein the signature score was produced from normalised levels of at least 25 biomarkers, wherein the at least 25 biomarkers were selected from a fifth group consisting of ACY1, COL5A1, RNPEP, ABRACL, AK2, CALU, CDC37, CNDP2, CNPY2, COPE, COX6B1, CTSB, GAPDH, GRB2, GSTO1, HNRNPA1, HNRNPD, HNRNPF, HSPA4, HSPA9, IDH1, IDH2, LDHA, MDH2, MYL6, NPM1, P4HB, PGK1, RCN1, RRBP1, S100A4, SET and VASP.

13. The method as claimed in claim 1, wherein the signature score was produced from normalised levels of all of the 33 biomarkers ACY1, COL5A1, RNPEP, ABRACL, AK2, CALU, CDC37, CNDP2, CNPY2, COPE, COX6B1, CTSB, GAPDH, GRB2, GSTO1, HNRNPA1, HNRNPD, HNRNPF, HSPA4, HSPA9, IDH1, IDH2, LDHA, MDH2, MYL6, NPM1, P4HB, PGK1, RCN1, RRBP1, S100A4, SET and VASP.

14. The method as claimed in claim 1, wherein the biological sample is:
(i) a sample of blood, serum or plasma; or
(ii) a sample of whole tissue breast tumour.

15. The method as claimed in claim 1, wherein the biomarkers are:
(i) proteins; or
(ii) RNA, preferably mRNA.

16. The method as claimed in claim 1, wherein the levels of the biomarkers are normalised against the levels of one or more control or house-keeping genes or proteins.

17. The method as claimed in claim 16, wherein the levels of the biomarkers are normalised by:
(i) subtracting the level of one or more control or house-keeping genes or proteins from the obtained levels of each selected biomarker; or
(ii) dividing the obtained levels of each of the selected biomarkers by the level of one or more control or house-keeping genes or proteins.

18. The method as claimed in claim 1, wherein the signature score is produced by summing the normalised levels of each of the selected biomarkers.

19. A method of obtaining an indication of the prognosis of breast cancer in a subject, the method comprising the step:
(a) producing a signature score from normalised levels of at least 3 biomarkers, wherein the at least 3 biomarkers are selected from a first group consisting of GAPDH, HSPA4, LDHA and VASP,
wherein the selected biomarkers were obtained from a biological sample which was obtained from the subject; and
wherein the produced signature score is indicative of the prognosis of breast cancer in the subject.

20. A method of classifying breast tumours, the method comprising the steps:
(a) producing a signature score from normalised levels of at least 3 biomarkers, wherein the at least 3 biomarkers are selected from a first group consisting of GAPDH, HSPA4, LDHA and VASP,
and wherein the selected biomarkers were obtained from a biological sample which was obtained from the subject; and
(b) classifying the breast tumour based on the signature score produced.
